(19) United States
(12) Patent Application Publication
(10) Pub. No.: US 2024/0361310 A1
(43) Pub. Date: Oct. 31, 2024

(54) Capture Beads for Use in Assays

(71) Applicant: Zafrens Inc., San Diego, CA (US)

(72) Inventors: Kandaswamy Vijayan, San Diego, CA (US); Ravi Ramenani, San Diego, CA (US); Chulmin Choi, San Diego, CA (US); Aurelien Laguerre, La Jolla, CA (US); Gopichandran Ravichandran, San Diego, CA (US); Nicholas Sam-Soon, San Diego, CA (US); Yi Zhang, San Diego, CA (US); David Milliken, San Diego, CA (US); Kenneth Chng, San Diego, CA (US); Maina Ndungu, San Diego, CA (US)

(73) Assignee: Zafrens Inc., San Diego, CA (US)

(21) Appl. No.: 18/734,732

(22) Filed: Jun. 5, 2024

Related U.S. Application Data
(63) Continuation-in-part of application No. 18/195,049, filed on May 9, 2023.
(63) Continuation of application No. PCT/US24/32014, filed on May 31, 2024.
(60) Provisional application No. 63/624,181, filed on Jan. 23, 2024.
(60) Provisional application No. 63/339,782, filed on May 9, 2022.

(51) Int. Cl. G01N 33/545; G01N 33/53; G01N 33/543; G01N 33/58

(52) U.S. Cl. CPC G01N 33/545; G01N 33/5308; G01N 33/54313; G01N 33/582; G01N 33/588

(57) ABSTRACT

Disclosed are capture beads that are suitable for use in an assay device which is configured to correlate the capture bead to a specific examination area in the device as well as correlating any captured nucleic acid or other cellular components generated during the assay to that bead.

### FIELD

This disclosure provides for a capture bead that is suitable for use in an assay device that is configured to correlate the capture bead to a specific examination area in the device as well as correlating any captured nucleic acid or other cellular components generated during the assay to that bead. This disclosure further provides for methods, devices, and systems that include said capture bead.

### BACKGROUND

Capture beads can be used in assays to isolate certain cellular components generated by an assay that is desired for analysis after assay completion. In general, assay devices have a multiplicity of examination areas that allow for thousands to millions of assays to be conducted simultaneously in one device. Examination areas in the device can be populated with one or more cells of interest and a separate assay can be conducted in each examination area. The assay typically includes a perturbation element to perturb the cell or cells and then determines the consequences that the perturbation had on the cell or cells. A capture bead is included in the examination area. When the assay is complete and the cell is lysed, nucleic acids released from the cell are then captured by the bead. Typically, but not necessarily, a single perturbation element is combined with at least one cell that is to be perturbed. After the capture of the nucleic acid(s), the capture bead is isolated, and the captured nucleic acids are released and then sequenced.

In one conventional system, the assay employs a perturbation bead that comprises multiple copies of the same perturbation element (e.g., compound or perturbation compound herein) which are releasably attached to the bead. In addition, the perturbation bead includes multiple copies of an oligonucleotide releasably bound to the bead. This oligonucleotide is generated during the synthesis of the compound on the bead and records the reaction steps and/or building blocks used to synthesis the compound. This oligonucleotide is sometimes referred to herein as the “perturbation oligonucleotide”. In some embodiments, the perturbation element is a compound, and the perturbation oligonucleotide is sometimes referred to as a “compound oligonucleotide”. The assay is conducted by releasing the perturbation compound from the perturbation bead thereby perturbing the cell. After completion of the assay, the cell is lysed. When the compound oligonucleotide includes one or more nucleic acid capture elements, the nucleic acids released from the cell (after lysis) are captured regardless of whether the compound oligonucleotide is released from the bead into the examination area or retained on the bead. In some embodiments, the nucleic acid is mRNA and the nucleic acid capture element is poly-T.

By attaching poly-T to the perturbation oligonucleotide, mRNA released from the lysed cell is captured by the perturbation oligonucleotide due to its poly-A tail. In some embodiments, the perturbation oligonucleotide comprises a poly-T and mRNA bound thereto that can be sequenced in a single sequencing step on the bead. The resulting information provides insight into the changes in functionality of the cell induced by the perturbation as well as the structure of the perturbation compound based on the reactions conducted to make this compound. More likely, the compound oligonucleotide is released from the bead and then captures the mRNA as solution phase capturing by hybridization is more efficient than hybridization involving a solid bound oligonucleotide and a solution phase mRNA.

To obtain more meaningful information from these assays such as those changes in functionality of the cell occurring during the assay which are induced by the perturbation both on a static and on a dynamic (e.g., real-time) basis, it is necessary to identify the changes in cellular functionality generated during the assay and then correlate these changes with the changes in mRNA expression ascertained only by sequencing after completion of the assay. However, this requires correlating the oligonucleotides used to capture the mRNA back to the capture bead from which they were initially bound and then back to the specific examination area where the assay was run and, hence, the cell in that examination area.

Limiting the amount of information just to static changes in cellular functionality generated by a perturbation to just the mRNA and the perturbation compound that generated the perturbation also limits the value of the information so collected. Other information regarding changes in cellular functionality during the assay that are left unaddressed include, by way of example only, the specific well from which the capture bead was retrieved, changes in cellular morphology, how the assay conditions affect the changes in cellular functionality, as well as other information such as the technician conducting the assay, the date of the assay, and other perturbation elements that might be captured.

However, all or some of this additional information is often not collected during an assay for a variety of reasons including the inability to correlate the mRNA generated by the perturbation compound back to the specific examination area where the assay was conducted, and any observations of the cell therein. Information regarding the specific examination area which generated the nucleic acid is lost as is any information regarding other functional changes in the cell itself. This prevents association with a more complete description of the changes in functionality of the cell and a more complete understanding of the impact of the perturbation element on the cell. Adding such information provides a more complete picture of how the perturbation altered the functionality of the cell and, whether such changes were deemed to be positive or negative.

Accordingly, there is an ongoing need to expand the amount of information retrieved from one or more of the examination areas found in an assay of a library of diverse compounds. Such additional information would expand the knowledge obtained from each assay and would facilitate drug discovery.

### SUMMARY

This disclosure provides for capture beads that are configured to collect information on both static and dynamic changes in cellular functionality generated during an assay and induced by exposing a cell to a perturbation element. In some embodiments, the information collected by the capture beads described herein correlates the change in functionality in a cell generated by the perturbation element to the examination area where that cell was perturbed.

The ability to generate such information is dependent on the ability to correlate the sequencing data generated by the perturbed cell back to the examination area where the cell was deposited prior to the start of the assay.

In some embodiments, there is provided a capture bead for use in an assay, said capture bead comprises:
a) a detectible label or a set of detectible labels attached to said bead which uniquely identifies the capture bead;
b) a multiplicity of a capture bead index which codes for the detectible label or set of labels which is unique to said capture bead and is attached thereto optionally through a linker; and
c) a multiplicity of one or more capture elements attached to the capture bead index.

In some embodiments, there is provided a mapped assay device comprising a multiplicity of examination areas and at least one capture bead in each of the examination areas in use in the device, wherein said mapped assay device correlates a given examination area to a specific location on said map and the capture bead(s) included in each examination area, wherein said capture bead comprises:
a) a detectible label or a set of detectible labels, attached to said bead, that uniquely identifies the capture bead;
b) a multiplicity of a capture bead index that codes for the detectible label or set of labels, is unique to said capture bead and is attached thereto optionally through a linker; and
c) at least one capture element attached to the capture bead index.

In some embodiments, the detectible label or set of detectible labels used in said capture beads is/are optically detectible.

In some embodiments, the detectible labels or sets of labels comprise different colors and/or the different intensities of one or more colors that are associated with different of said capture beads.

In some embodiments, the detectible label or set of labels is selected from the group consisting of images, codes, shapes, colors, inducible colors, lettering, numbering, symbols, bar codes, universal product code (UPC), and materials associated with the capture bead.

In some embodiments, the optional linker on said capture bead is a releasable linker.

In some embodiments, the capture elements comprise from about 6 to about 2,000 nucleotides.

In some embodiments, the capture elements attached to the multiplicity of the same capture bead index are selected from a receptor or ligand for the perturbation component to be captured or a complementary oligonucleotide to a corresponding oligonucleotide sequence contained in the perturbation oligonucleotide to be captured.

In some embodiments, the capture element is an oligonucleotide.

In some embodiments, a capture element, such as an oligonucleotide capture element, is attached to the proximal and/or distal end of the oligonucleotide index (where the proximal end is adjacent to the releasable linker or the bead).

In some embodiments, the attachment is either direct or through a linker.

In some embodiments, the linker is a releasable linker.

In some embodiments, the capture element is a receptor or ligand for the perturbation component to be captured. Such receptors include antibodies or antibody binding fragments for the perturbation component; an enzyme or enzyme receptor for the perturbation component, a receptor for a given cytokine or chemokine, and the like.

In some embodiments, the capture element on the capture bead is avidin/streptavidin.

In some embodiments, the capture element is biotin which captures perturbation components that have been modified to contain an avidin/streptavidin group.

In some embodiments, the capture bead index is attached to an oligonucleotide capture element which is attached either directly or through a releasable linker.

In some embodiments, the capture element is poly-T, poly-G, or poly-C.

In some embodiments, there is provide a capture bead as described above and represented by Formula I:

(W)ᵣ—CB-(L-X-Q-Q¹)ₙ (I)

where:
CB is a capture bead;
L is an optionally releasable linker;
W is an optically detectable label or a set of optically detectable labels that uniquely identify the capture bead;
X is a bond or a capture element;
Q is a capture bead index that codes the optically detectible label or set of optically detectible labels on the capture bead;
Q¹ is a capturing element which is independently selected for each of the n L-X-Q-Q¹ groups;
n represents the multiplicity of such (L-X-Q-Q¹) groups bound to the bead, and r is an integer from 0 to 100 (or more, depending on the size of the bead and type/size of the.

In some embodiments, r ranges from 10 to 6.02×10¹⁶. In some embodiments, n ranges from about 10⁵ to 6.02×10¹⁷.

In some embodiments, only multiple copies of the same single oligonucleotide index are bound to the capture bead.

In some embodiments, X and Q¹ are binding elements that capture different targets (e.g., target perturbation components, as may be released by a cell during an assay, and/or perturbation oligonucleotides as discussed herein).

In some embodiments, each X and Q¹ are independently selected for each of the n L-X-Q-Q¹ groups.

In some embodiments, each X and Q¹ are selected from a functionality that is complementary to a functionality found either on the perturbation oligonucleotide or on nucleic acids or other perturbation components released by the lysed cell.

In some embodiments, each Q¹ is an oligonucleotide.

In some embodiments, one of X and Q¹ captures a perturbation oligonucleotide and the other captures perturbation components.

In some embodiments, there is provided a capture bead having the Formula I-A:

(W)ᵣ—CB-[L′-X-Q-Q¹-Y]ₙ (I-A)

where:
Q is a capture bead index;
X is a bond or a capture element;
Y is a perturbation component PC or a perturbation oligonucleotide PO;
Q¹ is a capture element wherein, when X is not a bond, both X and Q¹ are independently capture elements wherein the capture element on each of Q¹ and X are complementary to a corresponding functional group on a target;
Y is a perturbation component PC or perturbation oligonucleotide;
L′ is a releasable linker;
and n, r, and W are as defined above.

In some embodiments, there is provide an oligonucleotide represented by the Formula I-B:

A-X¹-Q-Q¹-Y (I-B)

where:
PC is a perturbation component;
Q is the capture index;
Q¹ is a capture element complementary to a corresponding functional group on Y;
X¹ is a capture element complementary to a corresponding functional group on A;
Y is a perturbation component PC or a perturbation oligonucleotide PO; and
A is a perturbation component PC or the perturbation oligonucleotide PO.

In some embodiments, PC is mRNA and Q¹ is poly-T.

In some embodiments, A is a perturbation oligonucleotide and X¹ is a complementary group to a functional group on said perturbation oligonucleotide.

In some embodiments, A is a nucleic acid other than mRNA.

In some embodiments, a method is provided for associating sequence data from a solution phase capture index comprising one or more capture elements wherein at least one of said elements has bound thereto a perturbation component to a specific examination area in a mapped assay device which method comprises:
a) coding each of said capture beads through a releasable bond with an optically detectable label or a set of optically detectable labels unique to that bead wherein said capture bead comprises an oligonucleotide index that codes for said unique label or set of labels and further comprises a capture element thereon wherein said capture index is releasably bound to the capture bead;
b) adding a coded capture bead from a) to those examination areas in an assay device where an assay is to be conducted;
c) generating a registry which records the unique bead in each examination area;
d) conducting the assay in the presence of a cell which is perturbed during the assay;
e) lysing the cell and capturing at least one nucleic acid perturbation component onto the capture element;
f) releasing the capture index from the capture bead;
g) sequencing the capture index and the nucleic acid perturbation component to provide a correlation between the capture index and the labels on the capture bead; and
h) ascertaining the examination area that correlates to that unique label or set of labels by reference to the registry.

In some embodiments, the correlation is done with an image aligning each of the optically detectible capture beads to a site on the map.

In some embodiments, the optically detectable labels on said capture bead can include additional information that is related to the assay to be conducted in that examination area. The additional information includes, by way of example only, the conditions of the assay, the change in functionality of the cell during the assay, the synthetic reactions used in at least one step of the compound synthesis, the identity of one or more reaction conditions used during synthesis, the technician conducting the assay, the date of the assay, the pH of the assay solution, or combinations thereof. The unique optically detectable label on each of said capture beads in each examination area can be recorded by photography, bar codes, QR codes, and the like.

In some embodiments, such additional information can be included on the perturbation bead by a micro-component added to the examination area. Such micro-components coded with such additional information are described in U.S. patent application Ser. No. 18/195,049, entitled “High Throughput Single Cell Based Assay for Capturing Genomic Information for Functional and Imaging Analysis and Methods of Use”, filed May 9, 2023, which claims benefit to U.S. provisional patent application Ser. No. 63/339,782, filed May 9, 2024; International patent application Serial No. PCT/US24/23594, entitled “Cell Transfer Component for Multi-Well Assay Device”, filed Apr. 8, 2024, and/or U.S. provisional application Ser. No. 63/494,621, U.S. Provisional application Ser. No. 63/494,628 and U.S. provisional application Ser. No. 63/494,636, all filed on Apr. 6, 2023. Each of these applications is incorporated by reference herein in their entirety.

In some embodiments, the micro components can be of different shapes, same shapes but different colors, or different images such as different descriptions, barcodes, messages, QR codes, and the like placed thereon, In some embodiments, different micro-components are used in combination. In some embodiments, the different images are included on the same micro component such that the combination of such images provides for a unique micro component. See, e.g., FIG. 5C, where the micro component contains 4 quadrants each containing an image such as a drawing, written information, etc. The combination of these images is unique to that micro component.

In some embodiment, the unique micro component or combination of unique micro components provide(s) for a unique image that is uniquely associated with a given examination area. In some embodiments, each micro component used has a multiplicity of the same unique oligonucleotide strand associated therewith. In some embodiments, the unique strand is reversible attached to the micro component.

In some embodiments, the linker is used in combination with a unique perturbation bead. In some embodiments, the linker is reversibly attached to the micro component using a linker which is released in an orthogonal manner to the linkers used on the perturbation bead.

In some embodiments, the perturbation oligonucleotide on the perturbation bead codes only for the structure of the compound synthesized thereon. In some embodiments, the perturbation oligonucleotide comprises a binding element at its distal end. In some embodiments, the oligonucleotide strands on the micro components can have a complementary oligonucleotide binding element bound thereto that allows for hybridization to the binding element on the perturbation oligonucleotide.

In some embodiments, a single micro component having a unique label or set of labels thereon has a unique oligonucleotide strand that codes for the label and which is attached to the micro component by a cleavable linker with is cleaved in an orthogonal manner to the linker or linkers used on the bead. In a preferred embodiment, the oligonucleotide strand coding for such a micro component comprises a binding element that is complementary to a binding element on the distal end of the perturbation oligonucleotide such that upon release, the oligonucleotide strand from the micro component will hybridize to the binding element at the distal end of the perturbation oligonucleotide.

In some embodiment, multiple micro components are used such that the combination of these micro components is unique to an examination on an assay device. In some embodiments, the oligonucleotide strands on the micro component are capable of being hybridized to the perturbation oligonucleotide by use of unique combinations of binding elements on each component or by use of orthogonally cleaved linkers such that the oligonucleotide strands can be released and sequentially hybridized to the perturbation oligonucleotide.

In some embodiments, the micro components described above can be used in combination with a labeled or partially labeled perturbation bead that encodes or partially encodes for the compound synthesized thereon. In some embodiments, one or more steps of the reaction sequence used to synthesize the compound is/are coded by the micro component or a combination of micro components. In some embodiments, the unique set of micro components provides information regarding the reaction conditions and the like as described above.

In some embodiments, the unique micro component or combination of micro components are memorialized by photography or videography to correlate the examination areas where each unique micro component or combination of micro components are found. In some embodiments, the photography or videography is memorialized digitally.

Still further, the label or set of unique labels on said capture bead can be further compiled into said library or register thereby providing a complete set of information allowing the technician to correlate the capture bead to the specific examination area. Further, the perturbation component captured by the capture bead will be identified as originating from the specific capture bead by the capture bead index. In turn, the correlation between the detectible label and the examination area through the map will identify the examination area used with that capture bead. This allows for tracking the captured perturbation component back to the specific examination area from where it originated as well as to identify the perturbation element that induced the perturbation.

In some embodiments, only multiple copies of the same single capture bead index oligonucleotide are bound to the capture bead.

In some embodiments, X and Q¹ are the same.

In some embodiments, X and Q¹ are binding elements that capture different targets. In some embodiments, the capture of perturbation components and/or the perturbation oligonucleotide by X is accomplished after cleaving the releasable linker.

In some embodiments, the capture element comprises avidin or streptavidin and the perturbation component comprises biotin; or the capture element comprises biotin and the perturbation component comprises avidin or streptavidin. Similarly, the binding pair can be an enzyme and its binding partner, a cytokine or chemokine and its binding partner, an antibody/binding portion thereof, and its target.

When the capture elements on Q are oligonucleotides having bound thereto a perturbation oligonucleotide and/or a nucleic acid perturbation component such as mRNA, then the combination of X, Q, and Q¹ and the components captured by X¹ and Q¹ allow for sequencing multiple information components in a single step.

In some embodiments, the perturbation oligonucleotide further comprises an mRNA capture element such that after the assay is completed, Q¹ now has attached thereto the capture bead index-Q¹-perturbation oligonucleotide-mRNA. For example, attached to the distal terminus of the oligonucleotide index (Q) would be a poly-C group (Q¹) whereas the perturbation oligonucleotide could be constructed to have a poly-T group at one terminus and a poly-G group at the other terminus. When the perturbation oligonucleotide is released from the perturbation bead, the poly-G group can hybridize with the poly-C group of Q¹. When the cell is lysed, the poly-T group of the perturbation oligonucleotide will capture the poly-A tail of the mRNA released by the lysed cell.

In some embodiments, capture can be conducted either on the capture bead or in solution.

In some embodiments, the capture of a perturbation component is done on the capture bead. The capture bead is of Formula I-A:

(W)ᵣ—CB-[L-X-Q-Q¹-Y]ₙ (I-A)

where:
Q is a capture bead index;
X is a bond or a capture element;
Y is a perturbation component PC or a perturbation oligonucleotide PO;
Q¹ is a capture element wherein both X and Q¹ are independently capture elements when X is a capture element wherein the capture elements of both Q¹ and X are complementary to a corresponding functional group on the target (in this case for Q¹—a perturbation component);
and n, r, L, and W are as defined above.

In some embodiments, the linker is a releasable linker.

In some embodiments, when that linker is cleaved, either before or after capture of the perturbation oligonucleotide and/or the mRNA, the resulting oligonucleotide is represented by Formula I-B:

A-X¹-Q-Q¹-Y (I-B)

where each of Q, Q1, and Y are as defined above, X¹ is a capture element, and each of Y and A is independently a perturbation component or a perturbation oligonucleotide. In some embodiments, perturbation oligonucleotide is captured by Q1 prior to lysing the cell. Then, the releasable bond is cleaved allowing X¹ to capture a perturbation component, A, in the solution phase. Alternatively, the capture can be completely done in the solution.

In either event, the captured perturbation components are correlated to the capture bead by the capture bead index thereby identifying the examination area by reference to the registry.

In the above description, for each of the n L-X-Q-Q¹ groups, the identity of X and Q¹ can change/be different. However, in all cases, the capture bead index, Q, does not change as it has a unique correlation to the label or set of labels on the bead. In some embodiments, the perturbation element is a perturbation compound on a perturbation bead that is coded by the perturbation oligonucleotide. In some embodiments, there is provided a method for identifying a specific examination area in an assay device wherein a compound perturbation element is released, which method comprises:
a) conducting an assay in at least a portion of a multiplicity of examination areas in a mapped assay device wherein each of said examination areas comprises a cell, a perturbation bead comprising a perturbation element (e.g., compound), and a perturbation oligonucleotide which is unique to said perturbation element (e.g., compound), and an assay solution;
b) including in each of said examination areas a capture bead which comprises:
   an optically detectible label or a set of optically detectible labels, attached to said bead, which uniquely identifies the capture bead;
   a multiplicity of the same capture bead index which is unique to said capture bead and is attached to thereto optionally through a linker wherein said index codes for the unique label or set of labels; and
a multiplicity of a capture element attached to the oligonucleotide index;
c) correlating and memorializing the optically detectible label or set of labels in each examination area by associating the unique optically detectible labels on said capture bead to said mapped assay device thereby providing for a 1:1 relationship between each capture bead in each of the examination areas;
d) inducing a perturbation by releasing at least a portion of the perturbation elements from said perturbation bead thereby allowing said elements to contact said cell and induce functional changes in said cell which comprises at least a change evidenced by one or more perturbation components expressed by said cell;
e) releasing the unique perturbation oligonucleotide from said bead and capturing said perturbation oligonucleotide onto the capture bead;
f) lysing said cell to release said perturbation components into said assay solution and capturing at least one of said components onto the capture bead index or the perturbation oligonucleotide;
g) sequencing the oligonucleotide comprising the capture bead index, the perturbation oligonucleotide, and the perturbation component; and
h) correlating the capture bead index to a unique label or set of labels to a specific examination area by reference to the memorialized correlation in c) above thereby identifying the particular examination area from which the perturbation component was retrieved.

In some embodiments, the method provided above further comprises:
i) obtaining images of each of the examination areas during the assay;
j) correlating the images to the specific examination area defined in h) above; and
k) evaluating changes in cellular morphology and/or cellular function during the assay as a result of the perturbation of the cell.

In some embodiments, the perturbation element need not include a perturbation bead. Such perturbation elements may include, but are not limited to, one or more of the conditions of the assay such as, but not limited to, buffers, salts, pH, temperature, nutrients, oxygen levels, oxidizing agents, physical stress, and/or a presence of antibodies, immune cells, siRNA, viruses, bacteria, fungi, and the like. In such cases, the lack of a perturbation bead obviates the need for a perturbation oligonucleotide. Rather, a unique oligonucleotide can be associated with one or more of these different non-compound perturbation elements (e.g., by a releasable linker to a perturbation bead, and/or to the perturbation element itself in the case of immune cells, viruses, etc. and/or by adding to a corresponding examination area), such that the underlying principle remains the same—release (if releasably attached) of the perturbation oligonucleotide (i.e., coding for the perturbation element and/or condition) and capture by the capture bead will identify that perturbation element.

In some embodiments, the set of labels on said capture bead comprises two or more members. In some embodiments, the set of labels comprises from two to fifteen members.

In some embodiments, the set of labels comprises multiple members which, in combination, identify the capture bead to which they are bound.

In some embodiments, the perturbing element is a compound released from a perturbing bead.

In some embodiments, the capture bead is positioned in the examination area before, during, or after the completion of the assay.

In some embodiments, the capture bead described above is removed from the examination area prior to the removal of the oligonucleotides from the bead.

In some embodiments, the capture bead is added to the examination area prior to the assay. In some embodiments, the capture bead is added to the examination area during the assay. In some embodiments, the capture bead is added to the examination area after completion of the assay.

In some embodiments, there is provided a register for a given assay device, which register contains a record for each unique label or set of labels for each capture bead, the specific examination area where said capture bead is located in the assay device, and the unique oligonucleotide found on said capture bead.

In some embodiments, the register comprises one or more digital photographs that record the code associated with each examination area and the optically detectible label on the capture bead.

In some embodiments, the set of labels comprises labels having the same color but with different intensities.

In some embodiments, the digital photography uses megapixels wherein the record for each examination area comprises a different set of pixels from the other examination areas. The use of pixels allows for distinguishing fine shades of colors and also between the same color when used at different intensities.

In some embodiments, there is provided a method for conducting an assay in an assay device comprising a multiplicity of examination areas which method comprises:
A. combining into each examination area to be utilized:
   a) a perturbation bead comprising multiple copies of the same perturbation compound unique to that bead and a multiplicity of the same perturbation oligonucleotide that codes for said perturbation compound;
   b) a capture bead having the Formula I-A:

(W)ᵣ—CB-[L′-X-Q-Q¹-Y]ₙ (I-A)
where:
   Q is a capture bead index;
   X is a bond or a second capture element on Q;
   Q¹ is a capture element wherein both X and Q¹ are independently capture elements when X is a capture element wherein the capture element on both Q¹ and X are complementary to a corresponding functional group on a target;
   Y is a target, such as a perturbation component and/or a perturbation oligonucleotide (e.g., coding for a perturbation element, such as a perturbation compound released from a perturbation bead)
   L′ is a releasable linker;
   W is an optically detectible label or a set of optically detectible labels that uniquely identify the capture bead;
   n represents the multiplicity of such (L′-X-Q-Q¹) groups bound to the bead, and
   r is an integer from 0 to 100 (or larger, depending on the size of the capture bead and the type/size of label(s));
   c) a cell; and
   d) an assay solution;
B. initiating the assay by cleaving at least a portion of the releasable linkers on the perturbation bead thereby releasing at least a portion of the perturbation compounds and the perturbation oligonucleotides from each bead in each examination area;
C. capturing the perturbation oligonucleotide on the capture element on said capture bead, wherein said perturbation oligonucleotide optionally comprises or is modified to comprise a binding element for a perturbation component;
D. lysing the cell at the completion of the assay thereby releasing perturbation components from said cell, at least a portion of which are captured on the capture bead by X when it is a capture element or by Q¹ or by the perturbation oligonucleotide;
F. isolating the capture bead and cleaving the releasable bond, wherein the capture bead index and the targets captured thereon are released into the solution; and
G. sequencing the capture bead index and the oligonucleotide targets captured thereon.

In some embodiments, cleaving of the releasable linker from the capture bead does not occur until after the assay is completed, including lysing of the cell.

In some embodiments, cleaving of the linker on said capture bead is conducted after cleaving the linkers on the perturbation bead but prior to lysing the cell.

In some embodiments, cleaving the linker on the capture bead is conducted prior to or concurrent with the release of the perturbation oligonucleotide from the perturbation bead such that the capture of perturbation components and/or the perturbation oligonucleotide is conducted in the assay solution.

In some embodiments, there is provided a method for preparing a population of capture beads wherein each bead in said population comprises a unique label or a set of labels and a capture bead index specific for that label or set of labels which method comprises:
a) selecting a population of beads which are functionalized with an oligonucleotide binding site;
b) splitting said beads in approximately equal numbers into a multiplicity of reaction vessels, wherein the number of reaction vessels can be any integer (e.g., up to 12);
c) into each reaction vessel, applying the same first label to each of the beads in a given reaction vessel, wherein the label used in each vessel is different from labels used in the other vessels;
d) attaching an oligonucleotide strand to said oligonucleotide binding site, wherein the oligonucleotide strand is the same for each of the beads in a common vessel, but different among the different vessels;
e) pooling the beads and mixing until homogeneous;
f) repeat the process of b) through e) a sufficient number of times such that substantially each of the beads in said population is uniquely labeled and substantially each bead comprises a unique capture bead index which comprises oligonucleotide strands attached to the prior oligonucleotide strands for each of the separate reaction steps.

In some embodiments, the population of such beads ranges from 10,000 to 10,000,000.

### BRIEF DESCRIPTION OF THE DRAWINGS

FIGS. 1A and 1B illustrate two different mapped assay devices having an orientation component.

FIG. 2A-2D illustrates perturbation beads (circle) comprising a set of optical labels attached thereto that uniquely identify each capture bead from other capture beads.

FIG. 3 illustrates the perturbation beads of FIGS. 2A-2D in a subset of examination areas depicted in FIG. 1B.

FIG. 4 illustrates a mixed bead population having beads of different colors and different color intensities for the same colors.

FIGS. 5A-5D show examples of labeling and labeled beads with micro components.

FIG. 6 illustrates the labeling area of a labeling device.

FIGS. 7A-7D show examples of beads labeled with micro components that correspond to compounds attached to the beads (e.g., perturbation compounds).

FIGS. 8A-8B show the results of an assay to determine compounds in a combinatorial library that actively perturb A549 cells.

### DETAILED DESCRIPTION

This disclosure provides, in part, for a capture bead that is suitable for use in an assay device that is configured to correlate the capture bead to a specific examination area in the device as well as correlating any captured nucleic acid or other cellular components generated during the assay to that bead.

### 1. TERMINOLOGY

In order to provide clarity to the reader, the following terms are defined. Terms that are explained have their scientifically accepted definition.

As used herein, the singular forms “a”, “an” and “the” are intended to include the plural forms as well, unless the context clearly indicates otherwise.

As used herein, the term “about” when used before a numerical designation, e.g., temperature, time, amount, concentration, and such other, including a range, indicates approximations that may vary by (+) or (−) 10%, 5%, 1%, or any subrange or a sub value therebetween. In one embodiment, the term “about” when used with regard to a dose amount means that the dose may vary by +/−10%.

As used herein, the term “comprising” or “comprises” is intended to mean that the compositions and methods include the recited elements, but not excluding others.

As used herein, the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure.

As used herein, the term “consisting of” shall mean excluding more than trace elements of other ingredients and substantial method steps. Embodiments defined by each of these transition terms are within the scope of this disclosure.

In addition, the following chemical/biology and related terminology is used:
a) assay device and mapped assay device;
b) associated therewith;
c) capture bead;
d) capture element or binding element;
e) capture bead index, capture index, capture oligonucleotide, or oligonucleotide index;
f) cell;
g) cellular components;
h) cellular morphology;
i) change in functionality;
j) complementary functionality;
k) compound;
l) detectible label or set of detectible labels;
m) examination area;
n) linker and releasable linker;
o) multiplicity or multiplicity of perturbation beads;
p) multiplicity of perturbation elements;
q) multiplicity of oligonucleotides or capture/binding elements;
r) nucleic acids;
s) oligonucleotide;
t) oligonucleotide strand;
u) optically detectible;
v) or a precursor thereof,
w) perturbation bead;
x) perturbation component;
y) perturbation compound;
z) perturbation element;
aa) perturbation oligonucleotide;
ab) releasably attached; and
ac) unique.

The meaning of these terms as used herein is provided below (in non-alphabetical order). Terms not included in the above list that are used herein have their accepted scientific meaning.

As used herein, the term “oligonucleotide strand” refers to oligonucleotides having from about 6 to about 20,000 nucleotides or from 16 to about 50,000 nucleotides. Such strands can be attached to each other to form an oligonucleotide.

As used herein, the term “oligonucleotide” refers to an oligonucleotide having from about 6 to about 500,000 nucleotides provided that if the oligonucleotide comprises 2 or more oligonucleotide strands attached together, then the oligonucleotide comprises from about 12 to about 500,000 individual nucleotides. It is understood that if an oligonucleotide is intended to be attached to another oligonucleotide, such is referred to as an oligonucleotide strand, as above. If, however, the oligonucleotide is to be used and/or discussed as a standalone component, then it is not referred to as a strand.

As used herein, the term “capture bead” or “CB” refers to any and all beads used in an assay to capture the perturbation oligonucleotide as well as one or more cellular components after perturbing the cell and then lysing the cell to release such cellular components (“perturbation components”). In some embodiments, the perturbation components include nucleic acids such as mRNA, DNA, and the like. In some cases, the perturbation components can be chemokines, cytokines, enzymes, proteins, glycoproteins, peptides, hormones, metabolites, protein complexes, protein-nucleic acid complexes, endogenous cellular ligands, and the like. One or more capture beads can be included in an examination area. Multiple capture beads can be used when different perturbation components are to be captured. Alternatively, a single capture bead is used such that different perturbation components are to be captured by that bead.

As used herein, the term “detectible labels” refers to any source of information that can be detected by visual, chemical, biological, audio, or other means to evidence the presence of the information generated by the label. In some cases, the detectible label generates or can be stimulated to generate a visual and/or optically detectable image/signal such as a color, visible light, lettering, numbers, symbols, barcodes, fluorescent particles, light emitting diodes (LEDs), micro-components such as microchips, UPCs, tags or combinations thereof that can be seen with an unaided eye or by use of a microscope and are sometimes referred to herein as “optically detectible labels”. In some cases, the detectible label generates chemical signals optionally requiring suitable instrumentation such as quantum dots, fluorescence (e.g., fluorescent particles, lasing particles, luminescent particles), mass spectra, nuclear magnetic spectra (e.g., H¹ or C¹³ spectrum), oligonucleotides, and the like. In some cases, the detectible signal generates an audio or electromagnetic signal such as those generated by a radio frequency identification device (RFID), WiFi, or a Bluetooth device. In some cases, the detectible label generates a biological signal or a biologically induced signal such as nucleic acids, bioluminescence, and the like. The particular detectible label or labels used in the embodiments disclosed herein is within the skill of the art being dependent on the constraints of the assays to be conducted and the need to uniquely identify a component or components in that assay. Combinations of different labels can be used.

As used herein, the term “unique” means that for a given perturbation bead having a perturbation element releasably attached thereto, there is a unique combination of that element with the label generated on the bead. That is not to say that every bead in the population of beads has a different perturbation element as the likelihood that there will be duplicate beads having the same unique perturbation element and label or set of labels exists because of probability considerations in the split-pool synthesis.

For example, in a split pool reaction scheme illustrated herein (e.g., reaction schemes A and B, as described herein), a population of 100,000 beads split into 10 groups of 10,000 beads each and then reacted in separate reaction vessels in step 1 with a label that is different for each reaction vessel would provide for 10 sets of beads with different labels. When pooled together and split again into 10 reaction vessels in step 2, the probability that a bead will be found in the same reaction vessel in step 2 as in step 1 is 10%. So, after step 2, the probability that beads will have the same two labels is now down to 1,000 for each reaction vessel or an aggregate of 10,000 duplications. Steps 3, 4, and 5 will likewise have a 10% probability of beads having been in the same reaction vessel throughout steps 1 to 5. Hence, for step 3, the probability is 10% of step 2 (10,000) or 1,000 out of 100,000. For step 4, that becomes 100 beads with the same likelihood of having duplicate labels, and for step 5, that number becomes 10 out of 100,000; and step 6, the probability is 1 out of 100,000. If desired, a step 7 and step 8 would reduce the number to less than 0.01 out of 100,000. At any point, after step 5, the limited probability of duplication has been reduced to the point that the overwhelming number of such beads are unique. For the sake of completion, the rate of duplication is can be less than 1 in 1,000, or can be 1 in 10,000, and also can be 1 in 100,000.

As used herein, the term an “examination area” or “area” is a defined area (e.g., coordinates, region, position, space, well, compartment, etc.) in an assay device, or portion of an assay device, where a single assay is performed at a given time. Multiple examination areas are employed in one or more assay devices. Examination areas may include droplets, wells, channels, and the like. Each area is capable of containing at least one cell, at least one capture bead, and at least one perturbation element in isolation from other areas and/or sets of areas (e.g., other locations on the assay device). In some embodiments, examination areas do not include points within a device that do not participate in an assay. That is to say that only those areas where an assay is conducted are deemed to be “examination areas” as discussed herein.

As used herein, the terms “capture bead index”, “capture index” “capture oligonucleotide index” and “an oligonucleotide index” are used interchangeably and refer to an oligonucleotide that codes for the unique label or set of labels on the capture bead thereby correlating the oligonucleotide index to the unique label or set of labels and then to the unique specific capture bead with that unique labeling. In some embodiments, the oligonucleotide index is attached to the capture bead optionally in a releasable manner. In some embodiments, the oligonucleotide index can be attached to a “capture element” that can capture a perturbation oligonucleotide and/or a perturbation component both as defined above. In some embodiments, the capture element can be a complementary group such as an oligonucleotide that can hybridize with a group of nucleic acids recovered from a lysed cell or with a portion of the perturbation oligonucleotide. In some embodiments, the capture element is a complementary functional group that recognizes and binds to a corresponding complementary functional group. Such complementary functional groups include avidin or streptavidin and biotin, an enzyme and its binding partner, a cytokine or chemokine and its binding partner, an antibody or the binding portion thereof, and its target. The complementary group that is captured by the capture element on the capture bead is sometimes referred to as the “target”.

In some embodiments, the capture bead index can be attached to the capture bead optionally through a linker including a releasable linker. In some embodiments, the oligonucleotide index includes a capture element at both its proximal and distal ends. In some embodiments, the capture bead and oligonucleotide index can be represented by the Formula I as defined above.

In some embodiments, the capture bead index is attached to a perturbation component (e.g., mRNA). In some embodiments, the capture bead index is attached to the perturbation oligonucleotide. In some embodiments, the capture bead index is attached to a perturbation component (e.g., mRNA) and the perturbation oligonucleotide in any order. In some embodiments, capture occurs during the assay and, in other embodiments, this occurs during and/or after the assay is completed. In some embodiments, a single capture bead is employed in each examination area. In some embodiments, multiple capture beads are employed in each examination area. In some embodiments, each of the capture beads in a single examination area is indexed with the same unique oligonucleotide specific for that capture bead. In some embodiments, each of the capture beads in a single examination area is indexed with a unique oligonucleotide index all of which correlate to that capture bead. For example, one or more of said additional capture beads are indexed with a unique capture element and a unique oligonucleotide index in order to capture different perturbation components generated by the perturbation.

As used herein, the term “perturbation oligonucleotide” refers to an oligonucleotide that uniquely codes for all or part of the perturbation element on the perturbation bead. In some embodiments, the perturbation oligonucleotide also codes for the unique label or set of labels on the perturbation bead, thereby uniquely identifying the perturbation bead. The perturbation oligonucleotide further comprises a complementary binding domain to the binding element on the capture bead. The complementary binding domain may be preferably either the proximal or distal portion of the perturbation oligonucleotide. In some embodiments, the perturbation oligonucleotide still further comprises or can be modified to comprise a complementary binding domain to the perturbation components released from the lysed cell. In some embodiments, the perturbation oligonucleotide is attached to the perturbation bead in a releasable manner. See, for example, U.S. Provisional Patent Application Ser. No. 63/624,167 and U.S. Provisional Patent Application Ser. No. 63/542,760, which are related to coding and/or partial coding by a perturbation oligonucleotide. These applications are incorporated herein by reference in their entirety.

As used herein, the term “perturbation element” refers to any source that perturbs a cell. Such sources include, by way of example only, peptides, compounds (e.g., drug-like small-molecules), antibodies, immune cells, siRNA, viruses, bacteria, fungi, change in one or more the conditions of the assay such but not limited to buffers, salts, pH, temperature, nutrients, oxygen levels, oxidizing agents, physical stress, and the like. In some embodiments, the perturbation element is a compound released from the perturbation bead. In some embodiments, a combination of two or more perturbation elements can be used. In some embodiments, the perturbing element is a compound released from a perturbation bead. Such a compound is sometimes referred to as a “perturbation compound” herein.

As used herein, the term “compound” or “perturbation compound” is an example of a perturbation element and refers to small molecules (having a molecular weight of less than about 15,000 Dalton), peptides, and/or oligonucleotides that are synthesized and bound to a perturbation bead through a releasable (cleavable) bond that is located between the compound and the bead. When the perturbation element is a compound on a perturbation bead that comprises multiple copies of the same compound releasable from the bead and multiple copies of a perturbation oligonucleotide which identifies the structure of or the identity of the compound, or the synthetic steps used to make the compound, the perturbation element is sometimes combined with the term “perturbation bead”.

As used herein, the term “perturbation component”, “perturbation product” or “PC” (or “PCⁱ” for integer i) refers to cellular components that are likely to be altered in quantity, expression, localization, identity, or other property upon exposure to a perturbation element. Such perturbation components may include mRNA, DNA, epigenetic modification, alternatively spliced mRNA, cytokines, chemokines, proteins (protein levels), protein isoforms, pre-mRNA, metabolites, lipids, glycoproteins, secreted proteins, and other such components that make up a cell. Embodiments of perturbation components as mRNA are primarily discussed herein for simplicity of explanation. However, any perturbation component (e.g., proteins, metabolites, etc., as listed previously) that can be represented by a nucleic acid barcode sequence may be used in addition to, or in place of mRNA as the nucleic acid perturbation components of this disclosure. Nucleic acid barcode sequences attached to antibodies or aptamers may be used to monitor protein levels and/or metabolite levels, with the antibodies or aptamers selected to bind to the target protein or metabolite of interest, and the barcodes are specific to the target protein or metabolite of interest. See, e.g., Stoeckius, M., et al. (2017). Large-scale simultaneous measurement of epitopes and transcriptomes in single cells. Nature methods, 14(9), 865. The nucleic acid barcodes representing the various perturbation components may be captured similar to mRNA as disclosed herein, e.g., via poly-A sequences on the barcodes hybridizing to poly-T segments as capture elements on the beads, or via other complementary sequences to other capture elements. In embodiments of this disclosure, perturbation components from a cell are analyzed after exposure of the cell to a perturbation element and after capture of the perturbation component on a capture-beads (or in some embodiments after capture on perturbation beads). In some embodiments, different types of perturbation components may be analyzed simultaneously. In such embodiments, the different and/or different types of perturbation components are denoted by PC¹, PC², and the like, to represent the diversity of perturbation components and/or types of perturbation components.

As used herein, the term “perturbation bead” refers to a bead that carries the perturbation element (e.g., in a releasable form). During an assay in an examination area, the perturbation element carried by the perturbation bead is released and allowed to interact with the cell in the examination area (e.g., the cell is exposed to the perturbation element). Typically, the perturbation bead carries multiple copies of the same perturbation element (e.g., perturbation compound) in a releasable form which compound constitutes the perturbation element. In some embodiments, the assay does not include a perturbation bead as the perturbation element constitutes one or more conditions of the assay as noted above or, alternatively, the perturbation element can be a virus, an antibody, or a cell such as an immune cell.

In some embodiments, the perturbation bead further comprises a perturbation oligonucleotide which codes for the perturbation element on said bead.

As used herein, the term “assay device” refers to a device having multiple examination areas for conducting assays using the capture beads as described herein.

As used herein, the term “mapped assay device” refers to any assay device where the examination areas can be identified by the geometry of the device (e.g., X and Y coordinates or any orientation that allows the individual examination areas to be identified such as triangular shapes, trapezoidal shapes, etc.) or by images, lettering, numbering, etc. associated with each examination area such that the examination area can be uniquely identified. In some cases, a mark, label, or other indicia on the assay device to allow the technician to properly orient the mapped assay device.

As used herein, the term “multiplicity of perturbation beads” or “multiplicity of perturbation beads” means more than one bead, such as at least 90 beads, or at least 1,000 beads, or at least 10,000 beads, or at least 100,000 beads. An upper limit of beads in all cases can be as many as 5,000,000 beads or more but generally is about 2,000,000.

The term “multiplicity” when used in conjunction with perturbation elements, oligonucleotides, binding elements, and the like means that there is a sufficient number of each to perturb a cell, to capture perturbation components from a lysed cell, and to assess the identity of the perturbation components as well as the perturbation element that generated the perturbation components, and the examination area from which the perturbation components were generated. In some embodiments, such a multiplicity ranges from about 1×10⁵ to about 6.02×10¹⁷.

As used herein, the term “releasably bound or attached” means that the specific component so described is attached to a capture bead in a releasable manner such that release/cleave from at least a portion of such compounds, oligonucleotides or nucleic acids from the bead can be initiated (e.g., by the technician) at an appropriate (e.g., desired, scheduled or controlled) time in the assay. Such controlled release is achieved in one instance by using a cleavable covalent bond—a bond that is cleaved under appropriate stimulation from light (e.g., ultraviolet light), heat, pH change, enzymatic activity, electromagnetic stimulation, sound, salt, change in oxidation, and the like. Such cleavable bonds are well-known in the art.

As used herein, the term “cellular morphology” refers to any one or a combination of two or more properties of a cell or components thereof such as the size, shape, structure, and/or form of a cell or components thereof. Cellular components are well known in the art and include the nucleus, the mitochondria, and ribosomes.

As used herein, the term “change in functionality” of a cell refers to one or more changes in cellular morphology as well as changes in the cellular expression of nucleic acids, proteins, chemokines, cytokines, enzymes, peptides, hormones, and the like as compared to the cell prior to exposure to the perturbation element. Other changes in functionality include the generation of apoptotic or other biological markers by cellular perturbation. Such apoptotic markers include chromatin condensation, caspase activation, blebbing, DNA fragmentation, and the like in the cell some of which can be readily visualized.

As used herein, the term “cell” refers to eukaryotic or prokaryotic cells. In some embodiments, the cell is a bacterial or yeast cell. In some embodiments, the cell is a primary cell or cell line for use in an assay. In some embodiments, the cell is a fish cell, amphibian cell, reptilian cell, avian cell, or mammalian cell. In some embodiments, the cell is, for example, a mammalian cell, such as a cell from a primate. In some embodiments, the cell is a human cell.

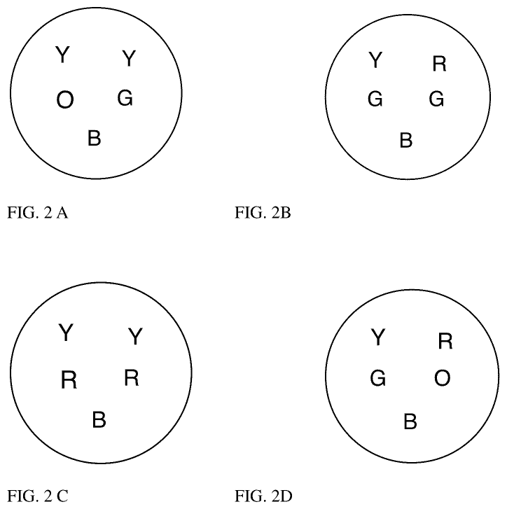

As used herein, the term “linker” refers to a group that attaches one entity to another. Such a linker is releasable or non-releasable. In some embodiments, the linker is a nucleotide or an oligonucleotide having from 1 to 2,000 nucleotide units. In some embodiments, the oligonucleotide incorporates a uracil (U) group which is cleaved by the User enzyme. In some embodiments, the oligonucleotide can be cleaved by a restriction enzyme. In some embodiments, the oligonucleotide is cleaved by a nicking enzyme. In some embodiments, clustered regularly interspaced short palindromic repeats (CRISPR®) technology is used to cleave the oligonucleotide. Chemical linkers such as carbonate, ester, and carbamate groups can be cleaved by pH, esterases, lipases, and the like. Photocleavable linkers are well known in the art and include coumarin groups, ortho-nitrobenzyl groups, and others found in, for example, FIG. 2 of Johan, et al., Pharmaceuticals (Basel), 15(6): 655 et seq. (June 2022). The selection of a suitable linker is not critical and is well-known to the skilled artisan.

In some embodiments, the linker is a non-nucleotide and comprises at least one and up to about 40 non-hydrogen atoms including carbon, nitrogen, oxygen, sulfur, and phosphorus. Where appropriate, these atoms include hydrogen (including all isotopes), hydroxy, oxo (═O), amino, or halo to satisfy the valence of the atoms. When the term “linker” is employed, unless stated otherwise or implicit from its use, it is intended to include direct attachment of the two groups without the introduction of any additional atoms.

The term “releasable” or “cleavable linker” means that the linker comprises a cleavable functionality—that is to say that the functionality or a covalent bond is readily cleaved into a first component and a second component by stimulation that breaks the bond. Such stimuli that can cleave a releasable bond include enzymes, UV light, heat, pH change, salt, and other components well-known in the art.

The term “static information” refers to changes in one or more cellular components that have changed and/or for which their expression has changed during the assay, and that are collected after the assay is completed. Such static information may include/be based on cellular components such as the expression of mRNA, upregulating or down regulating one or more cellular components that are captured by one or more capture beads such as chemokines, cytokines, hormones, enzymes, peptides, peptide fragments, and the like. Static information generally comprises and/or describes one or more, or all, of the molecular state of cells. Static information also includes perturbation components that are captured by the capture elements on the capture beads as well as perturbation components that can be analyzed directly in the lysed cellular milieu. For example, protein degradation components can be observed at the end of the assay but need not be captured to assess the perturbation components. In some embodiments, protein degradation components can be evaluated by immunofluorescent staining after the assay is completed if the perturbed and lysed cell is not from a stable cell line that contains innate fluorescence.

The term “dynamic information” refers to information regarding the cell as it occurs during the assay. Such dynamic information can include photo images of the cell undergoing perturbation including a picture(s) or a video of the perturbed cell during a portion or all of the assay. Such images evidence changes in cellular functionality such as changes in cellular morphology, movement, size, shape, adhesion, division, induction of apoptosis, gene expression (e.g., expression of fluorescent/fluorescently labeled proteins), intracellular organization/structure (e.g., cytoskeletal organization/structure, organelle structure), extracellular expression, evidence of other cellular structural/behavioral changes, and the like. For example, such dynamic information may include optically detectable evidence of the presence of apoptotic markers or other markers, protein upregulation or downregulation that can be monitored live and/or in real-time (e.g., fluorescent proteins, etc.), activation of T-cells thereby observing proliferation and change in their sizes, T-cells killing target cells, and the like. Such changes are deemed “live” or “real-time” as they are observed as occurring during the assay and can be recorded via pictures, video recording, three-dimensional analysis of the cell, and the like.

### 2. GENERAL DISCUSSION

This disclosure relates to assays comprising a multiplicity of individual assays conducted on the same assay device using a multiplicity of examination areas to ascertain certain properties or characteristics of cells in response to perturbation elements used in the assays. Such assays and assay devices are common in the art but typically were deficient in that the assays provide only collective, population-level, and/or static information regarding changes in the cellular contents such as nucleic acids expressed after perturbation of the cell after the assay was complete. However, dynamic information regarding the behavior of cells during the assay, static information at the single/few/small number (e.g., 10s of) cell(s) level and/or clearly linked to the dynamic information of corresponding single cells or direct connection between perturbation libraries and molecular responses of the cells was not available, as any correlation to the examination area and the perturbation was lost when the nucleic acids were sent for sequencing.

This disclosure provides methods and compositions that allow for the collection of static information as well as dynamic information that occurs in real-time which, when combined, provide a substantially more complete picture of the impact of the perturbation element on the cell.

The perturbations that alter cellular functionality include not only compounds but also the use of one or more pH changes, temperature changes, different assay conditions such as different buffers, nutrients, salts, hypoxic conditions, duration of the assay, and the like. Still further, the perturbation element can be an antibody, a cell such as an immune cell, or a collection of cells whose collective interactions influence each other as well as the cells of interest.

The methods and compositions now allow for a technician to correlate static and dynamic information by identifying the static information captured by the capture beads described herein which allows for ascertaining the exact examination area where the assay producing that static information was conducted. This then allows the technician to correlate the dynamic information collected for that examination area to the static information generated during the assay. When coupled, the static and dynamic changes in cellular functionality provide a more complete analysis of the totality of the functional changes generated by the perturbation. This is accomplished by including on the capture bead: a detectible label or a set of detectible labels that are unique to that capture bead; a binding element, a capture bead index reversibly associated with the capture bead that both captures perturbation components as well as correlating coded index back to the specific examination area when used with a mapped assay device.

By coupling each of these informational components, the technician can correlate the perturbation components captured by the capture bead using the index that is reversibly bound to the capture bead. The technician can then correlate the capture bead to a specific examination area using the map on the assay device. This then allows one to observe the dynamic changes in cellular functionality during the assay such as the changes in morphology and assign this to a particular capture bead by reference to photography or videography conducted on the examination area during the assay. Combining all or some of this information using knowledge as to what examination area was used, what perturbation element was used in that examination area, what capture bead was in that examination area, and what perturbation component was retrieved from that examination area by the capture bead is substantial static evidence of the changes in functionality. Still further, the photography or videography of the assay device taken during the assay provides the dynamic changes in cellular functionality occurring during the assay.

In some embodiments, if the perturbation element is a compound, the compound is bound to the perturbation bead by a releasable linker. In addition, a perturbation oligonucleotide is likewise releasably attached to the perturbation bead and identifies the compound bound to the bead. The one or more capture beads included in an examination area will capture the compound oligonucleotide as well as perturbation components released by the lysed cell thereby allowing the technician to ascertain the structure of the perturbation compound that was responsible for the perturbation of the cell captured both in a static and in real-time as well as the static changes in functionality generated by the perturbation compound (e.g., captured perturbation components).

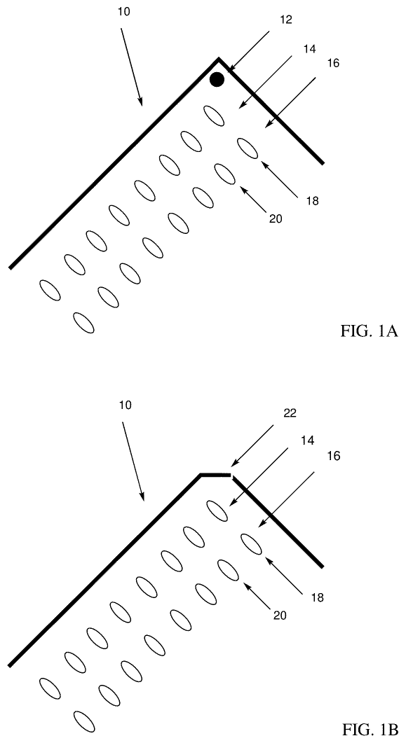

Specifically, an assay device comprising a multiplicity of examination areas is mapped such that each examination area is readily defined. Such mapping can include the X and Y coordinates for each examination area. Alternatively, each examination area in the device can be marked with a unique image or code. However, such is typically unnecessary when mapping the X and Y coordinates suffice. In those assay devices where the X and Y coordinates are ambiguous due to the lack of uniformity of the examination areas to each other, marking each area will resolve the ambiguity. Still further, an indicia or mark can be applied to the assay device to orient the device along its proper X and Y axis. See, for example, FIGS. 1A and 1B.

In addition, the methods described herein permit the use of multiple perturbations in an examination area. For example, multiple perturbations can include a first perturbation designed to generate a second perturbation and the assay measures the impact of the second perturbation on a cell of interest. Such can be illustrated by using a perturbation compound (first perturbation) to contact and perturb a T-cell which then contacts a tumor cell to determine the impact of the perturbed T-cell on the tumor cell.

In some embodiments, when the capture element attached to the oligonucleotide index on the capture bead is an oligonucleotide (“capture oligonucleotide”) then that capture oligonucleotide will capture either nucleic acid from the perturbed and lysed cell or the perturbation oligonucleotide released from the perturbation bead.

In some embodiments, the capture bead index on the capture bead comprises at least one capture oligonucleotide which will capture the perturbation oligonucleotide. If the capture bead index and/or the compound oligonucleotide comprises a second capture element or binding element, then, perturbation components can also be captured after the cell is lysed.

In some embodiments, the capture bead, CB, comprises a capture bead index (e.g., oligonucleotide index Q in Formula I) as well as a first binding element (e.g., Q1 in Formula I) and optionally a second binding element (e.g., X in Formula I) as shown in Formula I supra.

The combination of these otherwise separate informational components allows for real-time information generated during the assay to be associated with the cell used in that examination area. This allows the technician to combine the real-time and static information in a manner that provides significant information as to the change in cellular functionality that occurred during the assay.

### 3. PERTURBATION ELEMENTS COMPRISING A PERTURBATION COMPOUND

In some embodiments, the perturbation element is a compound releasably bound to a perturbation bead. Such perturbation compounds are synthesized on the beads using conventional split pool synthetic protocols well known in the art to generate a library of perturbation compounds. As per reaction scheme A below, which is provided for illustrative purposes only, a three-step synthesis for preparing a 1,000-member combinatorial library is depicted. Note that a four-step synthesis would provide for 10,000 possible compounds in the combinatorial library and a five-step synthesis would generate up to 100,000 compounds.

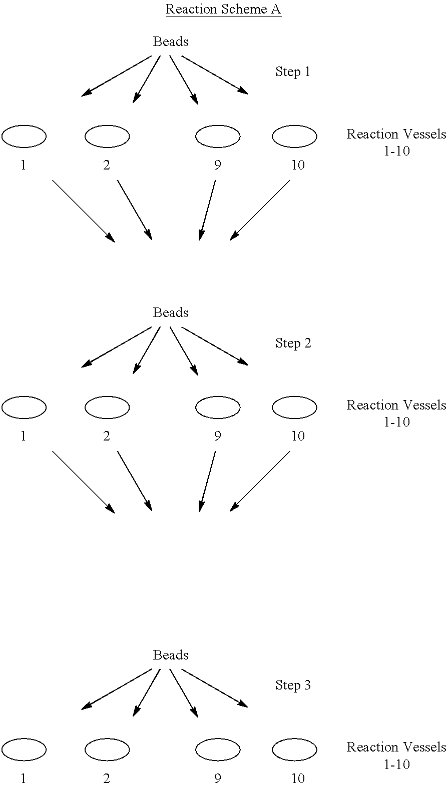

In step 1 of reaction scheme A, a multiplicity of beads (such as 1,000) are split into 10 vessels numbered 1-10 respectively (e.g., such that each vessel has approximately 100 beads). The beads comprise a cleavable linker for reacting with a component of interest to form the compounds that are to be made. In addition, there is an oligonucleotide site on the beads to which oligonucleotide strands can be added that define at least a portion of the structure of the perturbation compound to be generated thereon or the reaction steps used to make that compound (perturbation oligonucleotide—as per above). In some embodiments, the oligonucleotide site on the beads can contain a poly-C (cytosine), a poly-G (guanine), or a poly-T (thymine) having up to 2,000 nucleotides. This oligonucleotide site is distal from the linker and allows for oligonucleotide strands described below to be attached thereto.

In the case where the capture bead captures the perturbation oligonucleotide defining the structure or the reaction steps of the compound, the oligonucleotide site is bound to the bead by a releasable linker. In some embodiments, the releasable linker for the perturbation compound and the perturbation oligonucleotide are the same such that when the perturbation compound is released from the bead, so too is the compound oligonucleotide. The releasable linker may be cleaved under any means that cause the specific linker to break, reorganize, or otherwise release an attached compound/component (e.g., UV light exposure, pH, etc.). In some embodiments, the releasable linker used at the perturbation oligonucleotide site is orthogonal to the releasable linker used for the perturbation compound made on the bead. In some embodiments, the linker used for the oligonucleotide binding site is non-releasable under normal conditions including that used to cleave the perturbation compound from the bead.

Further in Scheme A, a first component of the perturbation compound to be synthesized can be added to the bead using chemistry that provides for very high yields (e.g., Click chemistry). The reactions can be run under conventional conditions that are maintained until the reactions are deemed complete. At this point, the beads comprise 10 different building blocks (e.g., beads in vessel 1 comprise a first building block in vessel 1, beads in vessel 2 comprise a second building block in vessel 2, and so on until the beads in vessel 10 comprise a tenth building block in vessel 10). An oligonucleotide strand can be introduced into each vessel either before, during, or after the reaction to memorialize the reaction performed in step 1 (e.g., oligonucleotide strands are coded by a sequence unique to each vessel, and each strand is connected to the preceding strand). Uniquely sequenced oligonucleotide strands in each reaction vessel can then be added to the bead, thereby memorializing and allowing for identifying the reaction conducted in that vessel.

In some embodiments, a first unique label or label is/are added to the perturbation bead in each reaction vessel by conventional methods such as click chemistry, amide bond formation, electrostatic binding, or other suitable conjugation chemistries familiar to trained chemists. Also, or alternatively, a label and/or labels may be added as described in International patent application Serial No. PCT/US24/23594, entitled “Molecular Library Encoding System and Methods”, filed Apr. 3, 2021, and/or International patent application Serial No. PCT/US24/23594, entitled “Cell Transfer Component for Multi-Well Assay Device”, filed Apr. 8, 2024, which applications, and applications from which they claim the benefit of priority, are incorporated by reference herein in their entirety. Alternatively, if the perturbation beads are provided as subsets, where each subset comprises a uniquely labeled bead distinct from beads in other subsets, then a single unique subset can be added to each vessel in the first step of the synthesis using approximately the same number of beads in each reaction vessel. This results in 10 reaction vessels that contain a population of beads where the beads in a given vessel all have the same unique label as compared to the labels on beads in the other vessels. In some embodiments, oligonucleotide coding the first step of the reaction in a given vessel and which is unique to that vessel will also code for the unique label used in that vessel.

In some embodiments, not all of the reaction steps in a split-pool synthesis need to be labeled. For example, in an 8-step synthetic process, a unique set of labels can be added to the perturbation bead by the sixth step. In such a case, the use of additional labels in the 7^{th} and 8^{th} steps becomes unnecessary and not conducted. So, in this embodiment, the addition of labels in the split-pool synthesis is terminated once a unique label or set of labels is added to the perturbation bead.

Generally, a unique oligonucleotide strand for each reaction vessel can be introduced either before, during, or after each reaction step and attached to the precursor strands to memorialize the reaction performed. During this step, a label or set of labels is added to each of the beads in a given vessel, and an oligonucleotide strand is attached to the precursor strand of oligonucleotide formed therein, which codes for the label and reaction conducted in or to be conducted in that vessel. Alternatively, as noted above, a separate oligonucleotide strand can be used to separately identify the label or labels added. In this case, it makes no difference if the first unique oligonucleotide strand coding for the label is added before or after the oligonucleotide coding for the reaction step.

Further in Reaction Scheme A, the first component of the perturbation compound to be synthesized can be added to the bead using chemistry that provides for very high yields (e.g., Click chemistry). The reactions can be run under conventional conditions that are maintained until the reactions are deemed complete. At this point, the beads comprise 10 different building blocks (e.g., beads in vessel 1 comprise a first building block in vessel 1, beads in vessel 2 comprise a second building block in vessel 2, and so on until the beads in vessel 10 comprise a tenth building block in vessel 10).

In step 2, all of the beads from each of the reaction vessels are typically washed and then pooled and mixed (e.g., shaken, stirred, etc.) to ensure homogeneity. The beads are then split into a second set of reaction vessels in the manner described above (e.g., resulting in an approximately equal number of beads in each vessel). Each reaction vessel will receive a portion of the beads previously subjected to reactions in vessels 1-10. As such, each reaction vessel will receive beads comprising each of the 10 building blocks (e.g., beads comprising the first building block, beads comprising the second building block, etc.). The reaction vessels for step 1 can be used for step 2 (e.g., after washing vessels and/or otherwise removing any reagents remaining from the prior step). The reactions conducted on the beads in each vessel are memorialized by the addition of another strand of an oligonucleotide unique to each reaction vessel. The oligonucleotide strand is attached to the prior oligonucleotide strand to form a single strand. The beads are then pooled and mixed as described above. At this point, the beads comprise up to 100 different building blocks (e.g., all combinations of the first 10 building blocks and the second 10 building blocks, in a case that the first 10 building blocks and the second 10 building blocks are mutually exclusive). Step 2 may be repeated as many times as desired to form the combinatorial library.

In step 3 (e.g., the final synthesis step), the beads are once again split and added to different reaction vessels as described above. The reaction in step 3 results in the generation of 1,000 compounds on the beads (e.g., if different components/reactions are performed in each vessel in each step). The generated library of compounds on the beads can be tested for activity with a cell, for example, in an assay where a single bead and at least one cell (e.g., up to 250 cells, up to 50 cells, around 20-30 cells, down to a single cell) are combined.

In some embodiments, the final step can utilize an oligonucleotide strand to identify the component added to the compound to be synthesized thereby identifying the compound or the reaction steps used. In another embodiment, and unlike steps 1-2, described above, no oligonucleotide is added to memorialize the reaction in step 3. Rather, the beads from each vessel are washed and optionally dried and then are placed into an isolated location on the assay device (i.e., beads from each vessel are transferred to separate locations on a single assay device or into separate assay devices, wherein the separate locations or separate assay devices each contain examination areas, such as wells or droplets, for conducting the assay). Alternatively, the beads from each vessel are placed in separate assay devices marked with the reaction vessel from which they were retrieved. This allows the technician to immediately identify the last step of the reaction used for those examination areas that evidence interesting results.

### 4. OTHER PERTURBATION ELEMENTS

Other perturbing elements that are useful in the assays described herein include agents such as antibodies, immune cells, cancer cells, antigen presenting cells (APCs), synthetically engineered cells, siRNA, peptides, viruses, bacteria, fungi, a change in one or more the conditions of the assay such but not limited to buffers, salts, pH, temperature, nutrients, oxygen levels, oxidizing agents, physical stress, and the like. Each of these can be used alone or in combination with one or more of such agents including a perturbation compound.

As to immune cells, their addition to an examination area allows for the evaluation of whether such a cell will perturb a cell such as a tumor cell in a therapeutic manner. In addition, the examination area also can contain a perturbation compound to evaluate whether that compound will perturb an immune cell that previously was either non-responsive or marginally responsive in a manner that renders the immune cell therapeutic to the tumor cell. Still further, the immune cell can be used alone or in combination with a perturbation compound to assess whether such attenuates or enhances the activity of that cell.

As to antibodies, the addition of a weakly reactive antibody into an examination area containing a tumor cell allows for the addition of a perturbation compound that perturbs the tumor cell. One can then evaluate whether the perturbation induced in the tumor cell enhances the therapeutic activity of the antibody.

As to engineered cells, these may comprise cells engineered to secrete membrane-associated agents capable of interacting with the test cells in the examination area. In some embodiments, the engineered cells may be engineered to secrete synthetic proteins, cytokines, or antibodies. In some embodiments, the engineered cells may be engineered to produce unique small molecules or metabolites. Engineered cells are the foundation of biotechnology and the various methods are well known to technicians engaged in the art of biotechnology and/or synthetic biology.

As to other perturbation elements, the use of conditions to perturb a cell provides valuable information as to whether the perturbation is beneficial or detrimental to the cell. Where conditions are detrimental, therapeutic agents that induce a similar perturbation to a diseased cell are appropriate candidates for inducing similar changes in vivo.

As above, combinations of perturbation elements can be used to ascertain the perturbation impact of such combinations as compared to the separate perturbation elements applied individually.

The salient feature of the perturbation beads is their ability to self-identify the perturbation element associated therewith via the perturbation oligonucleotide. Also, or alternatively, in a case that the perturbation beads comprise an optically detectible label that uniquely identifies the perturbation bead and/or its location in an examination area of an assay device, the perturbation bead may optionally comprise a label oligonucleotide, which may be part of or combined with the perturbation oligonucleotide thereby allowing sequencing of that oligonucleotide, once freed from the perturbation bead and captured by the capture bead (e.g., by capture elements X, Q¹, A, etc.) to self-identify the unique label and/or set of labels on that perturbation bead and/or the perturbation element provided thereby. This, in turn, allows the unique label or set of labels to be correlated to the examination area by reference to the registry.

### 5. CAPTURE BEADS

Capture beads, as described herein, are used to capture perturbation components generated by a perturbed cell during the assay as well as the perturbation oligonucleotide. In addition, the capture beads comprise a unique label or set of labels on each bead wherein the label or set of labels is coded onto the bead using a unique oligonucleotide (“capture bead index”). To capture perturbation components and the perturbation oligonucleotide, one or more capture elements are attached to the capture bead. Such capture elements capture perturbation components such as mRNA, DNA, nucleic acids, cytokines, chemokines, enzymes, proteins, hormones, and the like. Such a diverse selection of perturbation components not only assesses the changes generated by the perturbation as compared to an unperturbed cell but also assesses whether such changes are beneficial or not. In total, a technician can provide a more thorough evaluation of the changes in functionality of the cell due to the perturbation by assessing as many components as possible.

In some embodiments, the capture bead contains multiple copies of the same oligonucleotide that codes for the unique label or set of labels on the bead. Such capture beads can be prepared as shown in Reaction Scheme B:

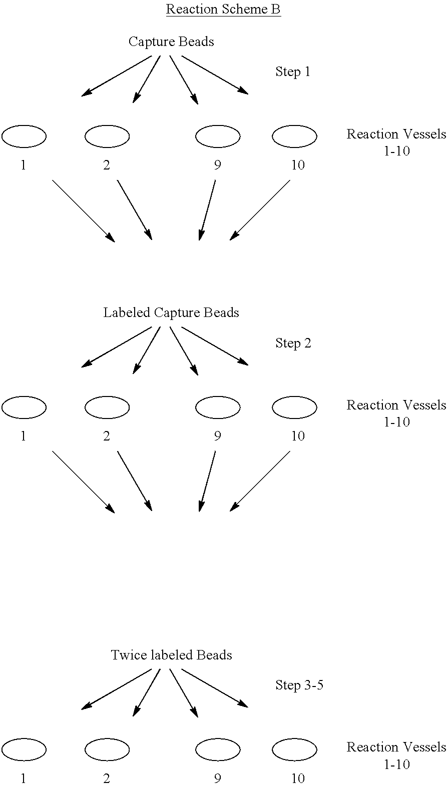

For example, in a 6-step process for labeling beads using a similar split-pool process with 10 reaction vessels and 100,000 beads where the beads contain a precursor oligonucleotide binding site which can contain a poly-C (cytosine), a poly-G (guanine), or a poly-T (thymine) having up to 2,000 members. This oligonucleotide site is distal from the linker and allows for oligonucleotide strands described below to be attached thereto.

In the first step of this scheme, the beads are split into substantially equal numbers of about 10,000 beads each. These beads are placed into separate reaction vessels labeled 1 to 10 and where each vessel employs a different label to label the beads. In some embodiments, a first unique label or labels is/are added to the perturbation bead in each reaction vessel by click chemistry and/or any methods, including conventional methods such as International Publication No. WO 2021/042011, the entire contents of which are incorporated herein by reference in its entirety.

Before step 2, the beads are homogenized and then split into the 10 reaction vessels in substantially equal numbers of about 10,000 beads each. Given that there are 10 vessels and a completely homogenized set of beads, the likelihood of beads from the same reaction vessel in step 1 being in the same reaction vessel in step 2 is 10% or about 1,000 beads are likely to be in each of the second reaction vessels as were also found in the same first reaction vessel. Using the same theoretical 10% reduction factor to assess the likelihood of duplication in each of steps 3-6, one can conclude that step 3 would reduce that aggregate number to 1,000 for all 10 vessels, step 4 would reduce that aggregate number to 100 (1 in 1,000), step 5 would reduce that aggregate number to 10 (1 in 10,000) and step 6 would reduce that number to 1 (1 in 100,000). In addition, at each step during the labeling process, a unique strand of an oligonucleotide that is common only to the same reaction vessel can be attached to an existing oligonucleotide strand on the beads such that after completion of the labeling, each unique set of labels on the beads is identified by a similarly unique oligonucleotide.

After step 6, there are 10 groups of about 10,000 beads each having the same label placed on the bead which indexes a particular set of labels on a given bead. At this point, the beads can be pooled and a common capture element can be attached. The beads are then represented by Formulas I and I-A above.

In addition, if the perturbation oligonucleotide (PO) is released when the perturbation elements are released into the examination area during an assay, then the perturbation oligonucleotides can also be captured by capture element on the capture indices on the capture bead. Such is depicted below in Formula II.

A-X-Q-Q¹-PC (II)

where A, PC, Q, Q¹, and X are as defined above.

In some embodiments, the capture bead is present during the entirety of the assay. If so, then at least one capture element on the capture bead is selected to capture the perturbation oligonucleotide. In some embodiments, one of the capture elements (X or Q¹) comprises a functionality that is complementary to a functionality found on the perturbation oligonucleotide. In some embodiments, the perturbation oligonucleotide will also have a second functionality that is complementary to a functionality on one or more of the perturbation components (e.g., perturbation nucleic acids) generated during the assay. The combination of both the perturbation components generated and the perturbation compound responsible for components is further information available to the technician.

When the perturbation component is mRNA, the capture element can be poly-T. As to cytokines and chemokines, binders such as antibodies or aptamers specific for these can be attached to the capture bead. Likewise, enzymes can be captured by a substrate to which they act upon and the like. Suffice it to note that the extent of targets to be captured by the capture bead is limited only by the recognition of a binding element specific to the target. In some embodiments, a binding element can be created for a particular perturbation component. For example, monoclonal antibodies can be generated by conventional means to bind to a specific perturbation component generated during the assay.

In some embodiments, multiple capture beads can be used in a single examination area. Each capture bead is directed to capture different perturbation components from the cell in the examination area. For illustrative purposes only, a first capture bead has bound thereto multiple copies of the same capture element for mRNA. A second capture bead has bound thereto multiple copies of the same capture element for a specific cytokine. Additional capture beads can be used to bind to other perturbation components. Each capture bead has bound thereto an oligonucleotide index unique to that capture bead. In some embodiments, the capture index comprises more than one capture element and, as such, can bind two different targets simultaneously. In some embodiments, the multiple capture beads may capture the same perturbation elements, the additional capturing elements allowing increased capture efficiency.

As to “W”, the optically detectible label(s) include one or more colors, induced colors, the same color at different intensities, lettering, numbering, images, insignia, barcodes, quantum dots, light emitting diodes (LEDs), QR codes and the like. A sufficient number of labels, r, are used, either alone or in combination, to uniquely identify that capture bead as compared to other capture beads used in the assay. This means that each capture bead to be used in an assay will be uniquely identified. In some embodiments, a single optically detectible label can be used such as a barcode or a QR code. In another embodiment, the capture bead can include multiple labels that in combination provide a unique code or image. Such embodiments include a mixture of colors, lettering and/or numbering, and the like. In some embodiment, “r” is an integer from 1 to 100 or from 1 to 10. In some embodiments, the capture bead is not labeled (r=0). Instead, the perturbation bead and/or examination area may comprise an optically detectable label or set of labels and a label oligonucleotide that codes for the label or set of labels and can be captured by the capture bead with the perturbation oligonucleotide and/or perturbation components.

As to n (as described above), it represents multiple copies of the linker bound components on the capture index on the capture bead. As used herein, the term “multiple copies” or “a multiplicity” as applied to bead-bound components means that the population of such bead-bound components that is 2 or more, 10 or more, 100 or more, and which can range up to about 1×10⁵ moles or 6.02×10¹⁷ individual molecules. The exact number is immaterial as long as there are sufficient amounts of the bead-bound materials to allow the assay to be conducted and to retrieve sufficient amounts of perturbation components to assess the cellular perturbations induced during the assay. In some embodiments, the number of attachments ranges from about 1×10⁵ to about 6.02×10¹⁷.

The specifics of capture beads suitable for use herein are not critical as long as the beads can be labeled and modified to include -L-Q-Q¹ or -L-X-Q and -L-X-Q-Q¹ groups as appropriate. In some embodiments, the capture beads can be polydimethylsiloxane (PDMS), polystyrene, glass, polypropylene, agarose, gelatin, hydrogel, paramagnetic, ceramic, plastic, methyl styrene, acrylic polymer, titanium, latex, Sepharose, cellulose, nylon, silicone, and any combination of the above. The particular material used for the capture bead is conditioned only on it being made of material that is inert to the assay to be conducted, including the solvent and reagents used.

In some embodiments, the size of the capture bead corresponds to a percentage of the size of the examination area where larger examination areas tolerate larger capture beads. In general, the size of the capture bead ranges from about 5 percent to about 50 percent of the size of the examination area (e.g., by volume). In absolute size, the capture bead is preferably from about 10 microns to about 250 microns along its longest axis and, more preferably from about 20 microns to about 200 microns along its longest axis, and even more preferably from about 30 to about 180 microns along its longest axis.

In some embodiments, the capture bead is added after assay completion where each of the capture elements as well as is releasable bound to the capture bead. By adding the capture bead after assay completion, any cleavable bonds used with the capture components can be the same as those used during the assay. Once the capture bead is recovered, the captured components bound to the bead can be released and then determined by conventional means.

Also, or alternatively, the capture bead can have bound thereto, optionally through a linker including a cleavable linker, a multiplicity capture elements such as biotin or avidin/streptavidin groups. For example, the perturbation oligonucleotide can be terminated with an oligonucleotide strand having a biotin or avidin/streptavidin group thereon optionally through a linker. The choice of each group is such that if a biotin group is either on the compound oligonucleotide or on the capture bead, then the avidin/streptavidin group is on the capture bead or on the compound oligonucleotide such that they are functionally complementary to each other.

In some alternative embodiments, one need not use an oligonucleotide to encode for the perturbation element (e.g., no perturbation oligonucleotide) or in the capture bead index. Rather, a compound having a known molecular weight and/or some other known/detectable characteristic can be used as the index on the capture bead. Such a compound would have a molecular weight that identifies the capture bead and is unique to other compounds used as an index on other capture beads. In some embodiments, the compound index is attached to the capture bead through a releasable linker. The index compound, once released, can be assessed by mass spectroscopy. In some embodiments, each of the index compounds on the different capture beads can be bound to the same material which increases its molecular weight. For example, the index compound can have a biotin group attached. Upon release, a compound having an avidin group can be bound to each of the compound indices so released. The resulting conjugate has an increased mass as compared to the index compound so that a mass spectrum of the conjugate is more readily done. In some embodiments, the index compound can be attached to the oligonucleotide by a dendrimer to increase its molecular weight.

### 6. REAL-TIME ANALYSIS

As above, this disclosure provides the technician with the ability to associate the perturbation component(s) generated by the perturbed cell to a specific capture bead that captured that perturbation component and then back to the examination area where the perturbed cell was located by reference to the map on the mapped assay device. In turn, by generating a record of each cell in each examination area that encompasses the assay period, one can access that record and assess the cellular changes that can be visibly observed as a result of the perturbation.

Such cellular changes include changes in morphology, movement, size, shape, adhesion, division, induction of apoptosis, gene expression (e.g., expression of fluorescent/fluorescently labeled proteins), intracellular organization/structure (e.g., cytoskeletal organization/structure, organelle structure), extracellular expression, evidence of other cellular structural/behavioral changes, and the like. Such changes are deemed “real-time” as they are observed as occurring during the assay and can be recorded via pictures, video recordation, three-dimensional analysis of the cell, and the like.

The technician can then combine the real-time changes in functionality with the static changes and provide a substantially improved overview of the cellular changes in functionality.

### 7. SYSTEMS

In some embodiments, a system is provided for providing information regarding an assay said system comprises:
a) a mapped assay device comprising multiple examination areas that are individually identifiable;
b) a perturbation bead comprising multiple copies of a perturbation compound releasably bound thereto which compound is unique to other perturbation beads to be used in the assay device and a perturbation oligonucleotide that codes for that compound;
c) a capture bead as described herein; and
d) a registry that correlates each capture bead to a specific examination area.

The systems described herein can further comprise one or more additional capture beads, one or more cells, or a combination of perturbation elements that act cooperatively (a first perturbation element that perturbs a second perturbation element that then perturbs a cell of interest). In some embodiments, sequencing of oligonucleotides bound to the capture bead can be done on the bead itself. In another embodiment, sequencing is done after releasing the oligonucleotides by cleaving the releasable bonds.

### 8. ASSAY PROTOCOLS

The assays described herein are conducted in a mapped assay device having a multiplicity of examination areas. For illustrative purposes only, the assays described herein employ a perturbation bead having multiple copies of the same perturbation compound releasably bound thereto and a compound oligonucleotide that memorializes all or part of the reaction steps used to make such compounds on a multiplicity of beads. Again, for illustrative purposes only, the compound oligonucleotide has attached thereto at its proximal end (relative to the releasable linker) a poly-T component that permits its capturing by a poly-A capture element on the capture bead. Alternatively, the compound oligonucleotide can have a first binding element at its proximal end and a second binding element at its distal end. For example, a poly-C compound could be attached to the proximal end and a poly-T component at the distal end. Upon cleavage of the releasable bond, the poly-C can hybridize to a poly-G group capture element attached to the capture index. In turn, upon cell lysis, the poly-T group can bind to mRNA.

In some embodiments, all or a portion of the examination areas in the assay device include a perturbation bead as described above, at least one cell to be perturbed (e.g., up to 250 cells, up to 50 cells, around 20-30 cells, down to a single cell), a capture bead as described herein including multiple copies of a capture bead index having a capture element attached thereto, and an aqueous solution suitable for conducting the assay. As above, the capture bead index and at least one capture element are attached to each other and to the capture bead in a manner where each attachment is optionally through a linker.

The identity of each perturbation bead in each examination area as well as the capture bead itself is memorialized or recorded by identifying the label on each capture bead and/or perturbation bead and corresponding that label to an examination area that is mapped by the assay device. This provides a 1:1 correlation between each examination area and each perturbation bead/capture bead maintained therein merely by referencing the unique label or set of labels found on the capture bead to the mapped examination area.

The assay is initiated by releasing at least a portion of the perturbation compound bound to the perturbation bead into the aqueous solution in a sufficient amount to perturb the cell. In some embodiments, the releasing mechanism (e.g., UV light) will also release the compound oligonucleotide from the perturbation bead as well as any releasable bonds on the capture bead that are responsive to release using the same releasing stimuli. In some embodiments, the perturbation oligonucleotide has a complementary functionality that is captured by the capture element on the capture index. For example, the complementary functionalities could be poly-A & poly-T, poly-G & poly-C, avidin/streptavidin & biotin, and the like. When the compound oligonucleotide is released, it will be captured by the complementary capture element on the capture index. The assay is then conducted for a sufficient time to induce one or more cellular perturbation(s). In some embodiments, photography or videography of the cellular response to the perturbation can be made. Upon completion of the assay, the cell is lysed to release cellular contents.

In some embodiments, the perturbation oligonucleotide further comprises a functional group that is complementary to nucleic acids released from the lysed cell. For example, the capture bead can have the Formula I-A:

(W)ᵣ—CB-[L′-X-Q-Q]ₙ (I-A)

as described above.

Upon release, the released nucleic acids (e.g., mRNA of the released cellular components and/or oligonucleotides released from perturbation beads such as the perturbation oligonucleotide and/or a label oligonucleotide of an optically detectable label on the perturbation bead) are captured by the complementary capture elements on the capture bead. The capture index on the capture bead now comprises the captured perturbation oligonucleotide which codes for the perturbation element. In some embodiments, the perturbation oligonucleotide comprises a capture element that captures perturbation components released from the lysed cell. In some embodiments, the capture bead is isolated from the examination area, washed, and prepared for sequencing. In some embodiments, the releasable bond attaching the capture index to the capture bead is cleaved releasing the capture index and the attached captured perturbation oligonucleotide, and the captured nucleic acids wherein each capture index can be sequenced where the sequence identifies the capture bead, the perturbation compound and the nucleic acids recovered from the lysed cell. In some embodiments, X is a capture element that can capture the same or different perturbation components released from the lysed cell. In turn, the identity of the labels on the capture bead index allows for a direct correlation to the examination area from where that bead originated by reference to the register recording the specific capture bead in the specific examination areas.

In some embodiments, some or all of the capture beads are pooled and the cleavable linkers on each are cleaved releasing a multiplicity of different oligonucleotides. Also, or alternatively, the oligonucleotides on the capture beads can be sequenced without release from the capture beads—polymerases may be employed to copy oligonucleotides attached to the capture beads into a complementary strand that is then sequenced. As each oligonucleotide is coded for the perturbation compound as well as the label or labels on the capture bead (and/or on the perturbation bead or otherwise in the examination area), each sequenced oligonucleotide can be associated with a particular capture bead and then to an examination area. Hence, each oligonucleotide to be sequenced will be self-identifying.

Once the capture bead identity is correlated to the examination area, a record of the functional changes in the cell in that examination area can now be directly correlated to the static changes evidenced by sequencing the lysed cellular contents.

In some embodiments, the order of attachment of the nucleic acids and the perturbation oligonucleotide is reversed such that the nucleic acids released from the lysed cell are first captured by the capture element on the capture index. In this embodiment, the perturbation oligonucleotide is bound to the perturbation bead by a releasable group that is orthogonal to the releasable group attaching the perturbation compound to the bead. When X is a capture element that is complementary to a capture element on the perturbation oligonucleotide, then, after the nucleic acids are captured, the perturbation oligonucleotide (poly-T) is released and captured by X. Afterwards, the perturbation oligonucleotide is released by cleaving the releasable bond which then allows for capture by the modified nucleic acids.

In some embodiments, recording or memorialization is conducted using digital photography or digital video recording. The digital aspects of such recordings allow for rapid assessment of colored labels used to uniquely identify a particular capture bead.

As noted previously, the capture bead can be added to the examination area before, during, or after the assay completion. However, the addition of the capture bead into the examination area before the start of the assay is preferred as it makes recordation of the examination area address to the particular capture bead easier if for no other reason than assay debris can be avoided when recordation is done.

### 9. EXAMPLES

The following examples are provided to illustrate the use of a capture bead as described herein. In this example, the following terms used therein have the following meanings:
bp=base pairs
poly-A=poly adenine
poly-T=poly thymine
mRNA=messenger RNA
UV=ultraviolet

### Example 1—Assay Device

A mapped assay device is employed which comprises a multiplicity of examination areas (e.g., picowells, nanowells, droplets, etc., as discussed herein). Each examination area is aligned in rows and columns such that the number of columns is longer than the number of rows. To orient the assay device, a mark, emblem, or other distinguishing feature can be added to one corner of the device to indicate an “up” position and/or standard orientation. FIG. 1A illustrates the arrangement of examination areas in a portion of a mapped assay device. Specifically, mapped assay device 10 has a mark, 12 in a corner of device 10 that directs the user to reference that corner as the upper left corner of device 10, for example. Any mark, indentation, or other feature can be used provided that it orients the device such that individual examination areas can be identified by columns 14, 16, and rows 18, 20. FIG. 1B shows an alternative means of orienting device 10, wherein an indentation 22 differentiates one corner of the assay device 10 from the others. In any case, a mapped assay device is provided where each examination area is identifiable by a column (e.g., 14, 16) and row (e.g., 20, 18). Any other distribution of examination areas may be used, as long as they can be unambiguously mapped.

### Example 2—A Library of Perturbation Beads

Step 1. A population of 100,000 beads having multiple copies of an oligonucleotide binding site and multiple copies of a compound synthesis site bound thereto are commercially available from Rapp Polymere GmbH, Ernst-Simon-Strasse 9, 72072 Tuebingen, Germany. The beads are divided in approximately equal numbers into 10 different reaction vessels. A unique first step of compound synthesis is conducted in each vessel such that each vessel generates a distinct building block different from the other building blocks generated in the other vessels. Either before, during, or after reaction completion, a first oligonucleotide strand that is unique to each reaction vessel is added to the oligonucleotide binding site such that all of the beads in the same reaction vessel will have the same first oligonucleotide strand which is different from the oligonucleotide strand found in the other vessels. After completion of the first step of the compound synthesis the beads are washed and then combined and homogenized.

Step 2. The procedure of Step 1 is repeated but using a unique second building block for compound synthesis in each of the 10 reaction vessels as well a unique second oligonucleotide strand for each reaction vessel. After completion of this step of the compound synthesis the beads are washed and then combined and homogenized.

Steps 3-5. The procedure of Step 2 is repeated but using a unique third, fourth, and fifth building block for compound synthesis as well as a unique third, fourth, and fifth oligonucleotide strand. After step 5, there are 100,000 different compounds made on the 100,000 different beads.

A single perturbation bead is then added to a single examination area in the asa device. Such a single perturbation bead can be illustrated as shown in Formula III:

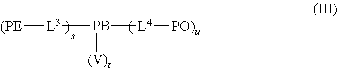

where the circle containing PB represents a perturbation bead, PE is a perturbation element, L³ and L⁴ are independently a releasable bond (cleavable bonds), PO is the perturbation oligonucleotide, V is an optional optically detectable label (e.g., in a case that the capture bead does not have such a label and/or r=0 in Formula I), and s, t and u are independent integers representing the numbers of bound groups to the perturbation bead. In some embodiments, s and u each be within the same value ranges as n above. In some embodiments, t can be within the same ranges as r as described herein.

### Example 3—Capture Bead

Each capture bead can be made from any starting material compatible with use in a cellular assay (e.g., any material inert to the cell and capable of having the groups/label(s) of, e.g., Formula I attached thereto or embedded therein), for example as described herein. The capture beads can each have a structure described by Formula I, for example. In an embodiment, each of the capture beads has bound thereto or embedded therein a set of labels comprising, for example, different colors, the same color but at different intensities, fluorescent particles that generate one or more colors, microchips, etc., or combinations thereof. A cleavable linker is attached to the bead or is found on the bead as provided. In some embodiments, a leader group for a capture bead index to be formed on the bead is attached to the linker. The leader group can be designed to be complementary to a capture element on the capture bead index. The capture bead index uniquely codes for the label or labels on the bead and is attached to the capture bead using methods well-known in the art. As above, each capture bead index is unique to the capture bead in a given examination area. In addition, although there are multiple copies of this index on the bead, no other oligonucleotides have a different sequence on the bead that codes for the label or set of labels. In this embodiment, the resulting capture bead can be represented by the following structure as provided in Formula IV:

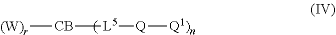

where CB represents the capture bead having a multiplicity of a capture element (Q¹) attached to a capture bead index Q which, in turn, is attached to the bead by a cleavable linker L⁵ (which may be as any cleavable/releasable linker described herein). L⁵ can further comprise X as described herein, resulting in Formula I. W represents one or more labels that uniquely identify the capture bead from other capture beads to be used in an assay device. Both r and n are as described herein.

In other embodiments, a second capture element can be included on the oligonucleotide index as shown in Formula V:

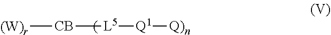

where r, CB, L⁵, Q, and Q¹ are as defined above. In Formulae IV and VI, for the n capture groups, each Q¹ can be the same or different from other Q¹s.

### Example 4—Labeling of capture beads and/or perturbation beads

FIGS. 2A-2D illustrate different optically detectible labels placed on a capture bead that uniquely distinguish that bead from other capture beads. (similar labels can be added to perturbation beads as described herein). Specifically, a unique combination of fluorescent dyes and/or their intensity of signal can be covalently attached to a capture bead such that the fluorescent signals generated can be associated specifically with that bead. In FIG. 2A, the first Y can be at one-third of the intensity of the second Y thereby distinguishing a first Y from a second Y. A large number of quantum dots are commercially available (from ThermoFisher, for example) and can be used for this purpose. The number of such dots required per capture bead is a function of the number of examination areas to be used in the assay device. For example, ten uniquely identifiable quantum dots can provide for over 3,000,000 unique combinations and/or signals. In FIGS. 2A-2D, B stands for blue; G stands for green, O stands for orange, R stands for red, and Y stands for yellow. In each case, the combination of colors using only these 5 colors provides for 120 unique combinations which is greatly expanded by adding just another 5 colors as above.

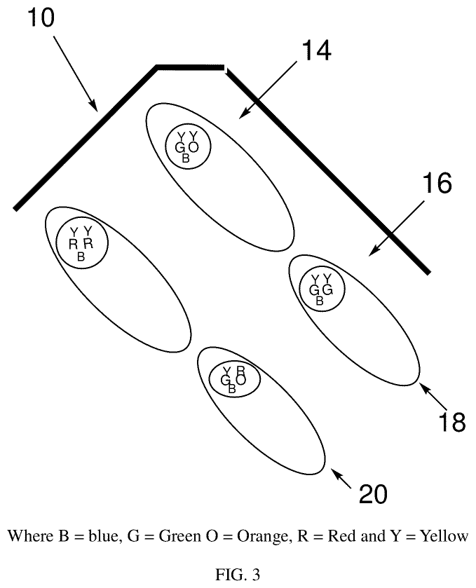

FIG. 3 illustrates an example of the placement of the labeled beads of FIGS. 2A-2D into separate examination areas as of an assay device 10 as illustrated in FIG. 1B.

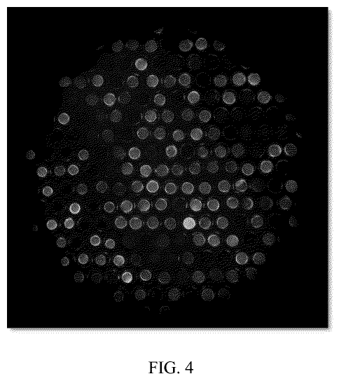

Also, or alternatively, perturbation beads and/or capture beads can be impregnated with and/or bound (e.g., covalently) to dyes such that each perturbation bead and/or capture bead has a distinguishable color or fluorescent-generated color that acts as a label and differentiates it from other perturbation beads. FIG. 4 illustrates an assay device with one bead per well, wherein each well contains optically distinguishable beads with different colors and intensities. In short, the beads are TENTAGEL™ beads that were formed by suspending one of:
1. 14 mg S30902 TENTAGEL™ resin, 10 ul QD600-WS quantum dot
2. 11 mg M30202 resin, 10 ul QD540-WS quantum dot, or
3. 11 mg M30352 resin, 10 ul QD680-WS quantum dot
   in 400 μL water for 20 minutes for equilibration in 1.5 mL Eppendorf tubes (quantum dots from NanoOptical Materials—CuInZnS/ZnS core/shell Quantum Dots with central emission peaks of 600 nm, 540 nm, and 680 nm, respectively). 4 mg 1-ethyl-3-(3-dimethylaminopropyl)carbodiimide (EDC) in 100 μL water was then added to each tube and allowed to react at room temperature for 16 hours with sporadic agitation. This resulted in TENTAGEL™ beads embedded with QDs emitting different colors and/or at different intensities such that each bead is uniquely distinguishable from the others by color and/or intensity.

Digital cameras recorded the color and intensity of the color by pixels. Accordingly, two dots of the same color but of different intensities can be readily distinguished by the pixels used to correlate differential intensities. This is readily done by computer analysis of the pixels.

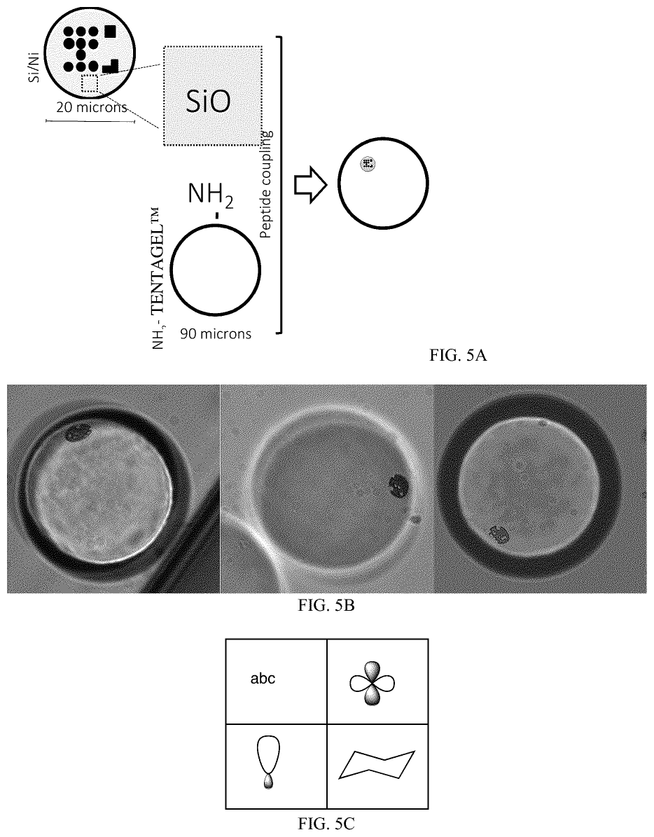

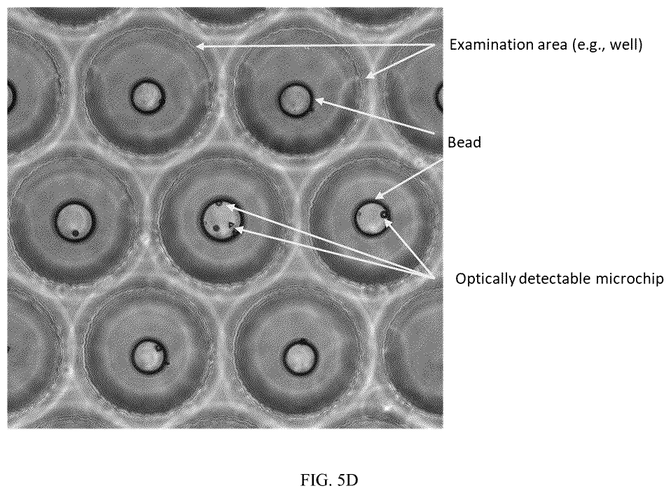

Also, or alternatively, perturbation beads and/or capture beads can be labeled with one or more micro-components that either have a detectable code thereon (e.g., FIG. 5A-B) or that form and/or constitute a detectable code (e.g., FIG. 5D) that uniquely labels the corresponding beads.

FIG. 5A shows a schematic for labeling a bead (in this example, a 90-micron TENTAGEL™ microbead—could be used as a perturbation or capture bead) with a micro-component (in this example, a silicon micropuck) having a visibly distinguishable code fabricated thereon. A layer of silicon oxide was deposited on silicon micropucks under ambient conditions, such that acidic silanols (SiOH) were formed on the surface of the micropucks. Silanols reacted readily with amine groups on the TENTAGEL™ beads (e.g., 90 micron TENTAGEL™ beads) resulting in stable electrostatic interactions that are stable under many solvents and reaction conditions. FIG. 5B shows three beads having encoded silicon micropucks stably attached thereto. The stability of the attachments of the micro-components to the beads (i.e., as in FIG. 5B formed as illustrated in 5A) was tested by incubating beads with attached micro-components in various chemicals/solvents for 24 hours. Beads were prepared as in FIG. 5A were incubated in 10% acetic acid in H₂O, Dimethylformamide, 10% ammonium hydroxide in H₂O, dimethyl sulfoxide, phosphate buffered saline, methanol, H₂O, or isopropyl alcohol for 24 hours. The micro-components remained attached to the beads in all conditions, demonstrating the suitability for the use of such beads in assays described herein.

Since the beads (and wells they are in) are transparent, the encoded micropuck can be imaged by brightfield microscopy regardless of orientation. The visibly distinguishable code is not particularly limited other than by the size of the micro-component and fabrication techniques. FIG. 5C shows an example of a 4-quadrant code, where each quadrant may include different information, potentially about the experiment and/or bead or conditions subject thereto.

FIG. 5D shows an example of microbeads with one or more optically detectable microchips attached thereto in individual examination areas (wells) of a multi-well assay device. FIG. 5D illustrates an example where the set and specific distribution of optically detectable microchips on each bead are unique to that bead. The optically detectible microchips in FIG. 5D are in a variety of shapes and sizes and were allowed to randomly attach to the beads, resulting in distinguishable random distributions of different sizes/shapes of microchips attached to beads. The beads and wells were transparent, making it possible to image the microchips on the beads by bright-field microscopy or other standard imaging techniques.

To prepare the beads in FIG. 5D, microfabricated silica microchips were first re-suspended in isopropanol at a concentration equivalent to ˜250,000 units per mL. 2 mL of the microchip suspension was gently mixed with 3.5 mg (approximately 168,000 units) of 30 microns NH₂—Tentagel perturbation/capture beads in a 15 mL falcon tube, and the mixture was rocked for 12 hours. The falcon tube was then spun down at 20,000 relative centrifugal force (rcf) for 2 minutes, and the supernatant was discarded. A fresh 2 mL of the microchip suspension was added to the beads, and the incubation and spinning were repeated once again. After the mixture was spun down the second time, the beads with random sets of microchips attached thereto were resuspended in pure isopropanol. The resulting optically detectable microchip-bead conjugates were stored at +4° C. until further loaded into examination areas. Once in the examination areas, the beads and the specific distribution of different microchips that uniquely identify the beads were imagable by brightfield microscopy, regardless of orientation due to the transparent beads.

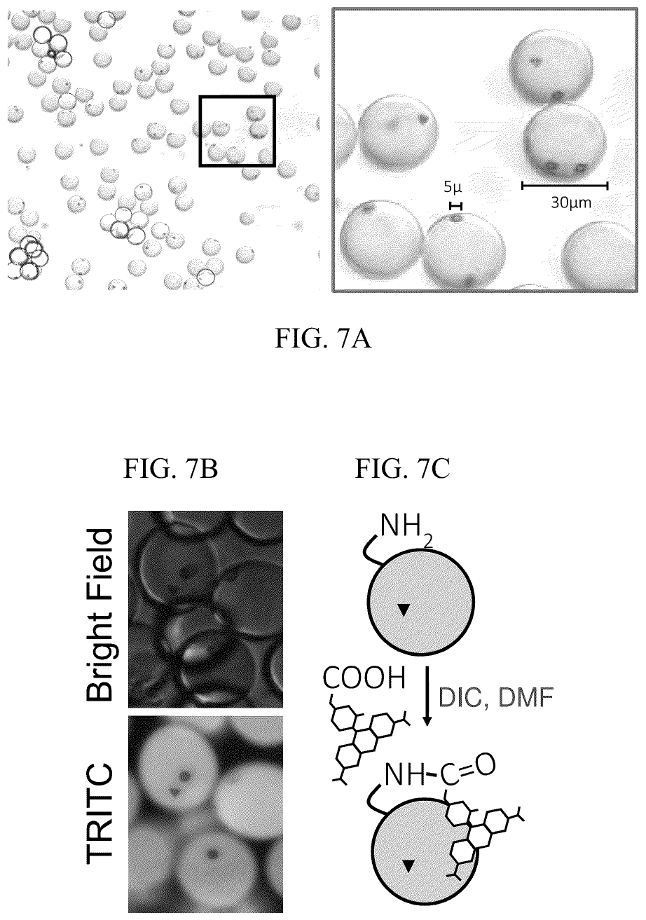

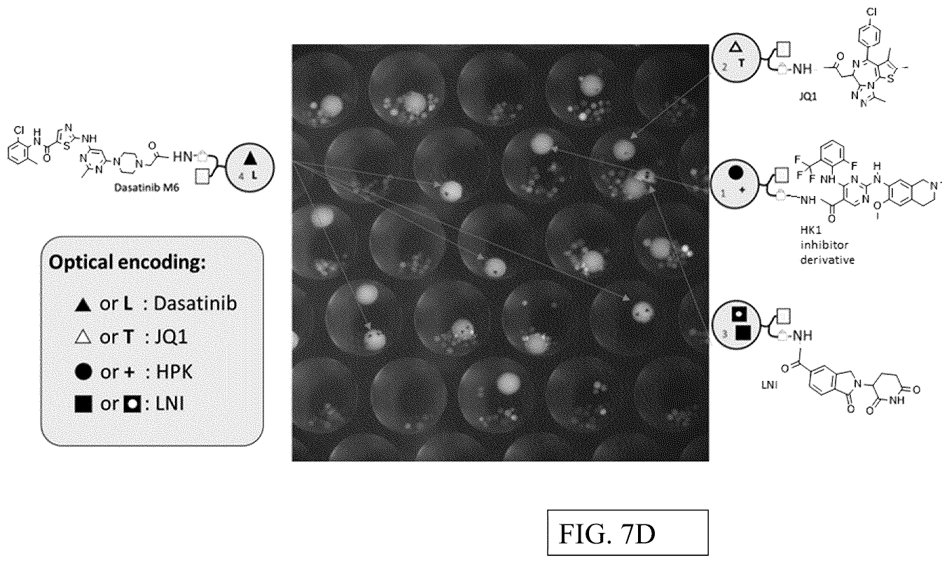

FIGS. 7A-7C show an example of adding labels (e.g., microchips) and corresponding compounds and/or compound building blocks to a bead (e.g., for preparation of a perturbation bead, for preparation of a capture bead that comprises a perturbation element, etc.). Beads shown in FIGS. 7A-7B were fabricated to have optically detectible microchips of different shapes randomly attached thereto. FIG. 7A shows a wide-view image (left) and a close-up image (right) of such beads in a reaction vessel (well of a 96-well plate). About 3.5 mg (˜168,000) of m NH₂-TENTAGEL™ capture beads were added to each well of a 96-well plate. To prepare resin with photocleavable linkers, TENTAGEL™ M NH₂, 10 μm dia. (dry), 10 mg, 0.23 mmol/g was loaded into a spin column and swelled in DMF (1 h, room temperature, RT). The resin was then reacted (3 h, 70° C., ×2) with 4-(Fmoc-aminomethyl)-3-nitrobenzoic acid (20 mM, 3.6 eq.) and N,N′-diisopropylcarbodiimide (DIC) (20 mM, 3.6 eq.) and washed with dimethylformamide (DMF) (3×0.6 mL). The fmoc-protected resin was deprotected with 20% piperidine in DMF (15 min, RT) and washed with dimethylacetamide (DMA).

To prepare the microchip-bead conjugates, microfabricated silica microchips of a given shape were first re-suspended in isopropanol at a concentration equivalent to ˜250,000 units per mL. Separate suspensions were prepared with unique shapes of microchips per tube. Then 2 mL of the microchip suspension was added to each well of the 96-well plate and gently mixed with the beads by rocking for 2 hours. The plate was spun down at 20,000 rcf for 2 minutes, and the supernatant was discarded. This operation was repeated twice. The medium was exchanged for dimethylacetamide (DMA). The previously added unique one or more micro components optically encode the building block information.

For peptide couplings used to conjugate the building blocks, standard conjugation conditions, and washes are employed, for instance, with the coupling performed with DIC (N,N′-diisopropylcarbodiimide) and with building blocks suspended in DMF (N,N-dimethylformamide). Detailed protocols of conjugation reaction are available in the supplementary information section of the publication https://doi.org/10.1021/ac500693r. Example building blocks used were: tetramethylrhodamine (TRITC) (FIGS. 7B-7C); Dasatinib metabolite m6, i.e., Dasatinib carboxylic acid—2-(4-(6-((5-((2-chloro-6-methylphenyl)carbamoyl)thiazol-2-yl)amino)-2-methylpyrimidim-4-yl)piperazin-1-yl)acetic acid; JQ1 acid—((S)-tert-butyl 2-(4-(4-chlorophenyl)-2,3,9-trimethyl-6H-thieno[3,2-f][1,2,4]triazolo[4,3-a] [1,4]diazepin-6-yl)acetic acid); HPK1 inhibitor derivative—4-{[2-Fluoro-6-(trifluoromethyl)phenyl]amino}-2-[(6-methoxy-2-methyl-1,2,3,4-tetrahydro-7-isoquinolinyl)amino]-5-pyrimidine-5-carboxylic acid; and LNI, lenalidomide acid—2-(2,6-dioxopiperidin-3-yl)-1-oxoisoindoline-5-carboxylic acid (FIG. 7D).

FIG. 7B shows brightfield and fluorescent images of beads formed as described above and, with triangular or circular microchips as optically detectible labels and tetramethylrhodamine (TRITC) as an example building block added to labeled beads as in FIG. 7C. FIG. 7B demonstrates sequential labeling of a bead with an optically detectable label, followed by the addition of a compound as an example building block without losing the optically detectable label.

FIG. 7D shows the results of adding beads prepared as above to have optically distinguishable labels corresponding to one of 4 building blocks/compounds (Dasatinib, JQ1, HPK, and LNI). Individual beads were added to wells of a multiwell assay device. Jurkat cells engineered to have enhanced green fluorescent protein (eGFP) reporter fused to the nuclear factor of activated T cells (NFAT) transcription factor were added to each well (cells purchased from BPS Bioscience, Catalog #78384). The compounds were released from the beads by exposure to 10 seconds of 365 nm wavelength light having an illumination power of 60 mW/cm² and allowed to interact with the cells in the respective wells. Cells exposed to JQ1, HPK, and LNI (identified by the corresponding optically detectible labels of the perturbation beads imaged in each cell) showed minimal change in the NFAT-eGFP expression levels, whereas cells exposed to Dasatinib showed almost complete downregulation of eGFP. This example demonstrates the use of optically detectible labels on the bead to code for perturbation compounds and/or building blocks and corresponding that visible code to a cell perturbation.

### Example 5—Assay Preparation

A cell (e.g., a human cell) is selected as the cell to be perturbed in the assay. The cell, the perturbation bead, and the capture bead are combined into a single examination area of a mapped assay device (e.g., as depicted in FIG. 1A or 1B) containing a suitable aqueous composition for conducting the assay. The combination of components provides for the following in a single examination well as shown in reaction Formula VI:

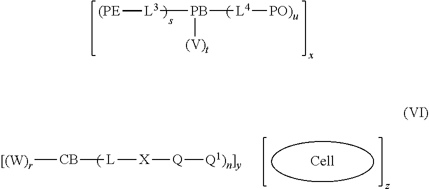

wherein L³, L⁴, L, PB, CB, PE, PO, V, W, X, Q, Q¹, s, t, u, r, and n are as described herein. Each of x, y, and z independently represents the number of each respective bead. In an example, each of x, y, and z are independently selected from 1-10. In addition, the ellipse with the term “cell” inside indicates an unperturbed cell. The perturbation bead, capture bead, and cell correspond to the examination area in which they are provided, and to each other.

After combining these components into an examination area of a mapped assay device also having other so-filled examination areas, then at any appropriate time during the assay but preferably before the start of the assay, a register is created by taking a digital picture of the assay device including each of the examination areas which register records the uniquely labeled capture bead in each examination area preferably in electronic form. In some embodiments, the picture is taken at a given depth and/or multiple depths and/or from multiple angles using confocal microscopy, for example. In some embodiments, the assay device is transparent, allowing for imaging from all angles.

The assay is initiated by releasing at least a portion of the perturbation compound from the perturbation bead into the assay solution to allow the perturbation compound to interface with the cell. When the compound oligonucleotide is attached to the perturbation bead via a releasable bond which, for the sake of illustration, is the same releasable bond attaching the perturbation compound to the perturbation bead, then application of an appropriate stimulus such as UV light will result in at least a portion of both the perturbation compound and the compound oligonucleotide being released into the assay solution.

### Example 6—Assay

Each of the assays conducted in the mapped assay device is incubated at standard conditions allowing the released compounds to perturb the cell. During the assay, pictures encompassing each assay well are taken at periodic intervals, or a video/digital recording is taken of the entire assay. Such pictures or video/digital recordings can evidence a change in cellular function or morphology as evidenced by a changed cell shape and other possible attributes as discussed elsewhere herein (i.e., dynamic information). Possible changes in cellular morphology are represented by Formula VII:

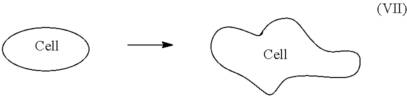

Here the original (solely illustrative) elliptical shape of the cell has been distorted into a shape evidencing, for example, blebbing and/or increased spreading presumably due to the perturbation caused by the release of the perturbation compounds. These changes captured during the assay are deemed to reflect dynamic functional changes in the perturbed cell and may be recorded in real time, e.g., by video and/or image capture.

Upon completion of the assay, the cell is lysed (dashed cell in reaction Scheme C, below) in the presence of the capture bead which captures perturbation components (PC) generated by the cell during the assay, as well as the perturbation oligonucleotide (PO) coding for the perturbation element (PE). Using the capture bead as described above, the perturbation components of interest in the cellular milieu are captured on the capture bead, as are the perturbation oligonucleotides as shown in reaction Scheme C below:

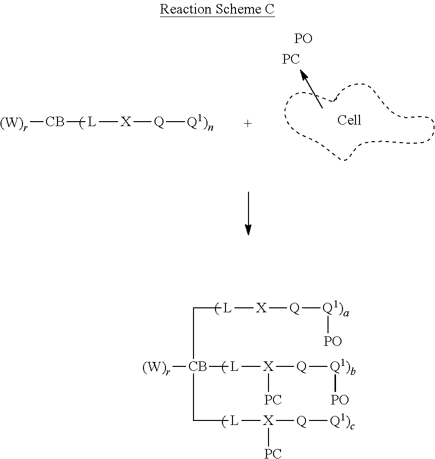

where all symbols are as defined herein, and where a, b, and c are each integers such that the sum of a, b, and c is less than or equal to n.

In the above reaction scheme, the perturbation compound and perturbation oligonucleotides are released and captured by the capture elements X and Q¹, respectively. releases the perturbation oligonucleotide, which is captured by the capture element attached to the oligonucleotide index on the capture bead. The assay is continued until completion and the cell in each examination area is lysed, releasing perturbation components. Reaction scheme C shows an example in which the L is not cleaved and/or is not cleavable, and capture occurs on the capture bead. In other embodiments, either before, during, or after lysing of the cells, L is cleaved, thereby allowing perturbation components and/or perturbation oligonucleotides to bind to capture elements X and Q¹ unhindered (e.g., fully in solution, without steric hindrance of the capture bead). Note that in the case where the perturbation component is mRNA, the entirety of the final product is a solution phase oligonucleotide suitable for sequencing.

In an example, A549 cells were screened against a Bromodomain Containing protein 4 (BRD4) focused, ˜10,000 member DNA-encoded (poly-T tailed) combinatorial library synthesized at the end of a nitro-benzyl linker attached to beads. Individual perturbation beads were deposited into corresponding micro-wells with a diameter of 130 m and 130 m depth, across a chip containing ˜47,000 wells. 5-10 cells were then added to each of the wells, which were then oil-capped with oil. Compounds were UV-cleaved from the beads to target a concentration of ˜5 μM and the chip was incubated for 24 hours in a 37° C. incubator. After the 24-hour incubation, the cells were lysed via freeze-thaw: −80° C. for 30 minutes and thawed for 45 minutes. The released mRNA was hybridized to the barcoded dT oligos on the same beads (perturbation bead acting simultaneously as capture bead). The oil caps were removed by washing the chip with a series of low-salt washes, protease digestion, and high-salt washes. The mRNA hybridized beads were removed from the chip and reverse transcription was performed. The beads were then split into eight whole-transcriptome amplification reactions. Libraries were generated via tagmentation of Nextera-B transposons and subsequent index-polymerase chain reaction (PCR). The captured nucleic acids from the cells were sequenced at ˜25,000 reads per well.

In a parallel chip, hits were identified by fluorescent microscopy. Briefly, cells were fixed at 4% paraformaldehyde (PFA) for 30 min at room temperature (RT) and permeabilized 0.5% triton x-100. Cells were blocked with 5% bovine serum albumin (BSA) with 0.2% triton for 40 minutes and then stained with BRD4 and B-tubulin antibodies for 2 hours at RT and subsequent secondary antibodies and Hoechst (nuclear) staining for 2 hours at RT. The chip was imaged and hits were identified by BRD4/tubulin vs BRD4/nuclear ratio. The “hits” were picked from the chip and amplified with Illumina P5/P7 primers. Libraries were sequenced at ˜10,000 reads per bead to identify the bead barcode/compound identifier oligonucleotide.

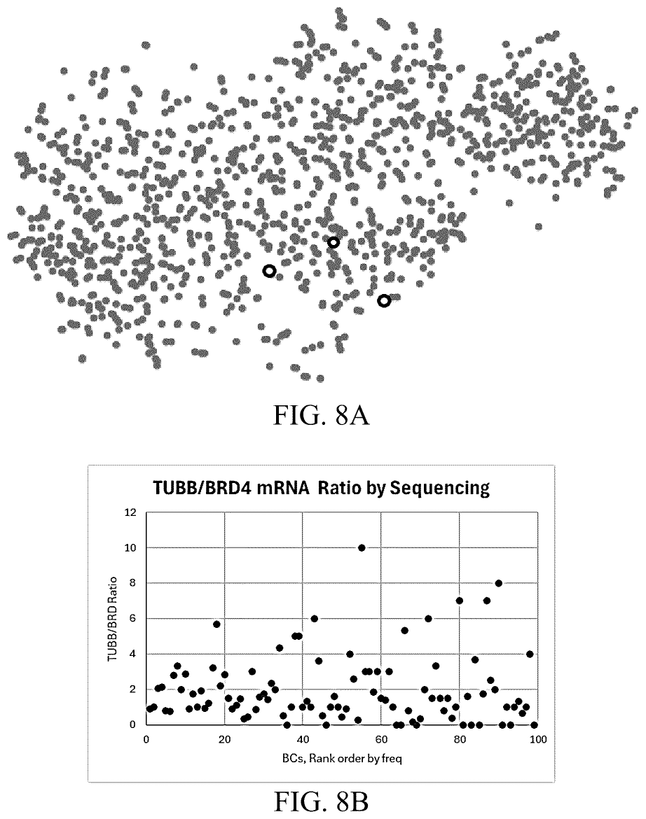

FIGS. 8A and 8B show the results of sequencing captured oligo from this example. mRNA-seq libraries were analyzed by first mapping using STAR (Systems Transcriptional Activity Reconstruction, e.g., Wang et al., Bioinformatics, 29(24): 3204-3210, December 2013, doi.org/10.1093/bioinformatics/btt558) on the hg38 annotated genome. Unique molecular identifiers (UMIs) were calculated to ensure accurate quantification of gene expression levels. Pre-processing steps were included to remove beads containing high mitochondrial and ribosomal content, and gene expression was normalized to the housekeeping genes. Dimensionality reduction techniques were applied to visualize the results on a two-dimensional plot based on transcriptomic similarity (t-distributed stochastic neighbor embedding plot in FIG. 8A. Hits identified from the fluorescent microscopy imaging were overlaid (see empty circles in FIG. 8A) and TUBB/BRD4 mRNA ratios calculated by sequencing (FIG. 8B). It is observed that compounds that cause decreased BRD4 levels by imaging upregulate BRD4 mRNA levels, suggesting a feedback loop that compensates for protein degradation by first increasing mRNA expression.

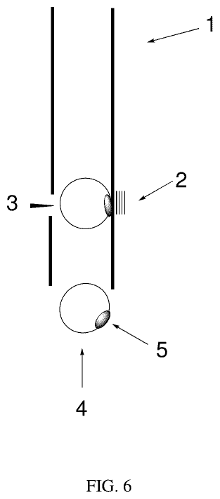

In some embodiments, the bead employed can be doped with a ferromagnetic material which can be used as an orienting component. For example, FIG. 6 illustrates a self-orienting aspect of a ferromagnet doped perturbation bead, 4, traveling up (or down) into the labeling site of shaft 1 of the labeling device (not shown).

In addition, perturbation beads added to the examination area can be oriented by placing a magnet under the assay device which then realigns the bead so that the labels are pointing substantially upward for imaging purposes.

Each of the assays conducted in the mapped assay device is incubated at standard conditions allowing the released compounds to perturb the cell. During the assay, pictures encompassing each assay well are taken at periodic intervals. Such pictures can evidence a change in cellular morphology as evidenced by a changed cell shape and other possible attributes. Possible changes in cellular morphology include distortions of cell shape. For example, if the cell has an original elliptical shape and is distorted by the perturbation into a shape that somewhat resembles an elliptical shape, but which also evidences areas of undulations and bulging due to the perturbation caused by the perturbation compounds. These changes captured during the assay are deemed to reflect dynamic functional changes in the perturbed cell.

Upon completion of the assay, the cell is lysed in the presence of the perturbation bead which captures perturbation components generated by the cell during the assay. Using the perturbation bead as described above, the perturbation components of interest in the cellular milieu are captured on the capture bead as are the compound oligonucleotides as shown in Reaction Scheme 2 above.

In some embodiments, each of the mRNA, the compound oligonucleotide, and the label oligonucleotide (if separate from the compound oligonucleotide) are sequenced either on the capture bead or separated from the capture bead by cleaving the cleavable bonds using an appropriate cleavage stimulation. The changes in the mRNA evidenced by the perturbation can be combined with any dynamic changes in cellular functionality as noted above to enhance the more of the changes in cellular functionality generated by the perturbation. The changes in nucleic acids expressed by the lysed cell can be correlated to the photography or videography conducted for the examination area from where the cell was lysed as described in detail above.

### 15. EMBODIMENTS

Each of the following embodiments is representative of a portion of the possible combinations that can be achieved herein many of which are explicitly provided above.

Embodiment 1: A capture bead for use in an assay conducted in an assay device said capture bead comprising a detectible label or a set of detectible labels attached to said bead which uniquely identifies the capture bead in said assay device.

Embodiment 2: A capture bead for use in an assay conducted in an examination area in a mapped assay device said capture bead comprising a detectible label or a set of detectible labels attached to said bead which uniquely identifies the capture bead in said assay device.

Embodiment 3: A capture bead for use in an assay conducted in an examination area in a mapped assay device comprising multiple examination areas, said capture bead comprising a detectible label or a set of detectible labels attached to said bead which uniquely identifies the capture bead in said assay device wherein the capture bead in said mapped assay device is correlated to a specific examination area thereby identifying that examination area where said capture bead is located.

Embodiment 4: A capture bead for use in an assay conducted in an examination area in a mapped assay device said capture bead comprising a detectible label or a set of detectible labels attached to said bead which uniquely identifies the capture bead in said assay device and said examination area in said mapped assay device is correlated to said capture bead wherein said correlation is memorialized.

Embodiment 5: A capture bead for use in an assay conducted in an examination area in a mapped assay device said capture bead comprising a detectible label or a set of detectible labels attached to said bead which uniquely identifies the capture bead in said assay device and said examination area in said mapped assay device is correlated to said capture bead and said correlation is memorialized into a register recording the position of each capture bead in each examination area in the mapped assay device.

Embodiment 6: A capture bead for use in an assay conducted in an assay device said capture bead comprising an optically detectible label or a set of optically detectible labels attached to said bead which uniquely identifies the capture bead in said assay device wherein said capture device further comprises multiple copies of a capture bead index that codes for the unique label or set of labels.

Embodiment 7: A capture bead for use in an assay conducted in an examination area in a mapped assay device said capture bead comprises an optically detectible label or a set of optically detectible labels attached to said bead which uniquely identifies the capture bead in said assay device wherein said capture device further comprises multiple copies of a capture bead index that codes for the unique label or set of labels.

Embodiment 8: A capture bead for use in an assay conducted in an examination area in a mapped assay device comprising multiple examination areas, said capture bead comprises an optically detectible label or a set of optically detectible labels attached to said bead that uniquely identifies the capture bead in said assay device wherein the capture bead in said mapped assay device is correlated to a specific examination area thereby identifying that examination area where said capture bead is located wherein said capture device further comprises multiple copies of an capture bead index that codes for the unique label or set of labels.

Embodiment 9: A capture bead for use in an assay conducted in an examination area in a mapped assay device said capture bead comprises an optically detectible label or a set of optically detectible labels attached to said bead which uniquely identifies the capture bead in said assay device and said examination area in said mapped assay device is correlated to said capture bead wherein said correlation is memorialized wherein said capture device further comprises multiple copies of a capture bead index that codes for the unique label or set of labels.

Embodiment 10: A capture bead for use in an assay conducted in an examination area in a mapped assay device said capture bead comprises an optically detectible label or a set of optically detectible labels attached to said bead which uniquely identifies the capture bead in said assay device and said examination area in said mapped assay device is correlated to said capture bead and said correlation is memorialized into a registry recording the position of each capture bead in each examination area in the mapped assay device wherein said capture device further comprises multiple copies of an capture bead index that codes for the unique label or set of labels.

Embodiment 11: A capture bead for use in an assay conducted in an assay device said capture bead comprising a detectible label or a set of detectible labels attached to said bead which uniquely identifies the capture bead in said assay device and a multiplicity of the same capture bead index which is unique to other capture bead indices used in said assay wherein said bead does not comprise other capture bead sequences coding for the label or set of labels.

Embodiment 12: A capture bead for use in an assay conducted in an examination area in a mapped assay device said capture bead comprising a detectible label or a set of detectible labels attached to said bead which uniquely identifies the capture bead in said assay device and a multiplicity of the same capture bead index which is unique to other capture bead indices used in said assay wherein said bead does not comprise other capture bead sequences coding for the label or set of labels.

Embodiment 13: A capture bead for use in an assay conducted in an examination area in a mapped assay device comprising multiple examination areas, said capture bead comprising a detectible label or a set of detectible labels attached to said bead which uniquely identifies the capture bead in said assay device wherein the capture bead in said mapped assay device is correlated to a specific examination area thereby identifying that examination area where said capture bead is located and a multiplicity of the same capture bead index which is unique to other capture bead indices used in said assay wherein said bead does not comprise other capture bead sequences coding for the label or set of labels.

Embodiment 14: A capture bead for use in an assay conducted in an examination area in a mapped assay device said capture bead comprising a detectible label or a set of detectible labels attached to said bead which uniquely identifies the capture bead in said assay device and said examination area in said mapped assay device is correlated to said capture bead wherein said correlation is memorialized and a multiplicity of the same capture bead index which is unique to other capture bead indices used in said assay wherein said bead does not comprise other capture bead sequences coding for the label or set of labels.

Embodiment 15: A capture bead for use in an assay conducted in an examination area in a mapped assay device said capture bead comprising a detectible label or a set of detectible labels attached to said bead which uniquely identifies the capture bead in said assay device and said examination area in said mapped assay device is correlated to said capture bead and said correlation is memorialized into a registry recording the position of each capture bead in each examination area in the mapped assay device and a multiplicity of the same capture bead index which is unique to other capture bead indices used in said assay wherein said bead does not comprise other capture bead sequences coding for the label or set of labels.

Embodiment 16: A capture bead for use in an assay conducted in an assay device said capture bead comprising an optically detectible label or a set of optically detectible labels attached to said bead which uniquely identifies the capture bead in said assay device and a multiplicity of the same capture bead index which is unique to other capture bead indices used in said assay wherein said bead does not comprise other capture bead sequences coding for the label or set of labels.

Embodiment 17: A capture bead for use in an assay conducted in an examination area in a mapped assay device said capture bead comprises an optically detectible label or a set of optically detectible labels attached to said bead which uniquely identifies the capture bead in said assay device and a multiplicity of the same capture bead index which is unique to other capture bead indices used in said assay wherein said bead does not comprise other capture bead sequences coding for the label or set of labels.

Embodiment 18: A capture bead for use in an assay conducted in an examination area in a mapped assay device comprising multiple examination areas, said capture bead comprises an optically detectible label or a set of optically detectible labels attached to said bead which uniquely identifies the capture bead in said assay device wherein the capture bead in said mapped assay device is correlated to a specific examination area thereby identifying that examination area where said capture bead is located and a multiplicity of the same capture bead index which is unique to other capture bead indices used in said assay wherein said bead does not comprise other capture bead sequences coding for the label or set of labels.

Embodiment 19: A capture bead for use in an assay conducted in an examination area in a mapped assay device said capture bead comprises an optically detectible label or a set of optically detectible labels attached to said bead which uniquely identifies the capture bead in said assay device and said examination area in said mapped assay device is correlated to said capture bead wherein said correlation is memorialized and a multiplicity of the same capture bead index which is unique to other capture bead indices used in said assay wherein said bead does not comprise other capture bead sequences coding for the label or set of labels.

Embodiment 20: A capture bead for use in an assay conducted in an examination area in a mapped assay device said capture bead comprises an optically detectible label or a set of optically detectible labels attached to said bead which uniquely identifies the capture bead in said assay device and said examination area in said mapped assay device is correlated to said capture bead and said correlation is memorialized into a registry recording the position of each capture bead in each examination area in the mapped assay device and a multiplicity of the same capture bead index which is unique to other capture bead indices used in said assay wherein said bead does not comprise other capture bead sequences coding for the label or set of labels.

Embodiment 21: A capture bead for use in an assay conducted in an assay device said capture bead comprising a detectible label or a set of detectible labels attached to said bead which uniquely identifies the capture bead in said assay device and a multiplicity of the same capture element on said bead.

Embodiment 22: A capture bead for use in an assay conducted in an examination area in a mapped assay device said capture bead comprising a detectible label or a set of detectible labels attached to said bead which uniquely identifies the capture bead in said assay device and a multiplicity of the same capture element on said bead.

Embodiment 23: A capture bead for use in an assay conducted in an examination area in a mapped assay device comprising multiple examination areas, said capture bead comprising a detectible label or a set of detectible labels attached to said bead which uniquely identifies the capture bead in said assay device wherein the capture bead in said mapped assay device is correlated to a specific examination area thereby identifying that examination area where said capture bead is located device and a multiplicity of the same capture element on said bead.

Embodiment 24: A capture bead for use in an assay conducted in an examination area in a mapped assay device said capture bead comprising a detectible label or a set of detectible labels attached to said bead which uniquely identifies the capture bead in said assay device and said examination area in said mapped assay device is correlated to said capture bead wherein said correlation is memorialized device and a multiplicity of the same capture element on said bead.

Embodiment 25: A capture bead for use in an assay conducted in an examination area in a mapped assay device said capture bead comprising a detectible label or a set of detectible labels attached to said bead which uniquely identifies the capture bead in said assay device and said examination area in said mapped assay device is correlated to said capture bead and said correlation is memorialized into a registry recording the position of each capture bead in each examination area in the mapped assay device and a multiplicity of the same capture element on said bead.

Embodiment 26: A capture bead for use in an assay conducted in an assay device said capture bead comprising an optically detectible label or a set of optically detectible labels attached to said bead which uniquely identifies the capture bead in said assay device and a multiplicity of the same capture element on said bead.

Embodiment 27: A capture bead for use in an assay conducted in an examination area in a mapped assay device said capture bead comprises an optically detectible label or a set of optically detectible labels attached to said bead which uniquely identifies the capture bead in said assay device. and a multiplicity of the same capture element on said bead.

Embodiment 28: A capture bead for use in an assay conducted in an examination area in a mapped assay device comprising multiple examination areas, said capture bead comprises an optically detectible label or a set of optically detectible labels attached to said bead that uniquely identifies the capture bead in said assay device wherein the capture bead in said mapped assay device is correlated to a specific examination area thereby identifying that examination area where said capture bead is located and a multiplicity of the same capture element on said bead.

Embodiment 29: A capture bead for use in an assay conducted in an examination area in a mapped assay device said capture bead comprises an optically detectible label or a set of optically detectible labels attached to said bead which uniquely identifies the capture bead in said assay device and said examination area in said mapped assay device is correlated to said capture bead wherein said correlation is memorialized and a multiplicity of the same capture element on said bead.

Embodiment 30: A capture bead for use in an assay conducted in an examination area in a mapped assay device said capture bead comprises an optically detectible label or a set of optically detectible labels attached to said bead which uniquely identifies the capture bead in said assay device and said examination area in said mapped assay device is correlated to said capture bead and said correlation is memorialized into a registry recording the position of each capture bead in each examination area in the mapped assay device and a multiplicity of the same capture element on said bead.

Embodiment 31: A capture bead for use in an assay conducted in an assay device said capture bead comprising a detectible label or a set of detectible labels attached to said bead which uniquely identifies the capture bead in said assay device and a multiplicity of the same capture bead index which is unique to other capture bead indices used in said assay and a multiplicity of a capture element attached to said index optionally through a linker.

Embodiment 32: A capture bead for use in an assay conducted in an examination area in a mapped assay device said capture bead comprising a detectible label or a set of detectible labels attached to said bead which uniquely identifies the capture bead in said assay device and a multiplicity of the same capture bead index which is unique to other capture bead indices used in said assay and a multiplicity a capture element attached to said index optionally through a linker.

Embodiment 33: A capture bead for use in an assay conducted in an examination area in a mapped assay device comprising multiple examination areas, said capture bead comprising a detectible label or a set of detectible labels attached to said bead which uniquely identifies the capture bead in said assay device wherein the capture bead in said mapped assay device is correlated to a specific examination area thereby identifying that examination area where said capture bead is located and a multiplicity of the same capture bead index which is unique to other capture bead indices used in said assay and a multiplicity of a capture element attached to said index optionally through a linker.

Embodiment 34: A capture bead for use in an assay conducted in an examination area in a mapped assay device said capture bead comprising a detectible label or a set of detectible labels attached to said bead which uniquely identifies the capture bead in said assay device and said examination area in said mapped assay device is correlated to said capture bead wherein said correlation is memorialized and a multiplicity of the same capture bead index which is unique to other capture bead indices used in said assay and a multiplicity of a capture element attached to said index optionally through a linker.

Embodiment 35: A capture bead for use in an assay conducted in an examination area in a mapped assay device said capture bead comprising a detectible label or a set of detectible labels attached to said bead which uniquely identifies the capture bead in said assay device and said examination area in said mapped assay device is correlated to said capture bead and said correlation is memorialized into a register recording the position of each capture bead in each examination area in the mapped assay device and a multiplicity of a capture element attached to said index optionally through a linker.

Embodiment 36: A capture bead for use in an assay conducted in an assay device said capture bead comprising an optically detectible label or a set of optically detectible labels attached to said bead which uniquely identifies the capture bead in said assay device and a multiplicity of the same capture bead index which is unique to other capture bead indices used in said assay and a multiplicity of a capture element attached to said index optionally through a linker.

Embodiment 37: A capture bead for use in an assay conducted in an examination area in a mapped assay device said capture bead comprises an optically detectible label or a set of optically detectible labels attached to said bead which uniquely identifies the capture bead in said assay device and a multiplicity of the same capture bead index which is unique to other capture bead indices used in said assay and a multiplicity of a capture element attached to said index optionally through a linker.

Embodiment 38: A capture bead for use in an assay conducted in an examination area in a mapped assay device comprising multiple examination areas, said capture bead comprises an optically detectible label or a set of optically detectible labels attached to said bead that uniquely identifies the capture bead in said assay device wherein the capture bead in said mapped assay device is correlated to a specific examination area thereby identifying that examination area where said capture bead is located and a multiplicity of the same capture bead index which is unique to other capture bead indices used in said assay and a multiplicity of a capture element attached to said index optionally through a linker.

Embodiment 39: A capture bead for use in an assay conducted in an examination area in a mapped assay device said capture bead comprises an optically detectible label or a set of optically detectible labels attached to said bead which uniquely identifies the capture bead in said assay device and said examination area in said mapped assay device is correlated to said capture bead wherein said correlation is memorialized and a multiplicity of the same capture bead index which is unique to other capture bead indices used in said assay and a multiplicity of a capture element attached to said index optionally through a linker.

Embodiment 40: A capture bead for use in an assay conducted in an examination area in a mapped assay device said capture bead comprises an optically detectible label or a set of optically detectible labels attached to said bead which uniquely identifies the capture bead in said assay device and said examination area in said mapped assay device is correlated to said capture bead and said correlation is memorialized into a register recording the position of each capture bead in each examination area in the mapped assay device and a multiplicity of the same capture bead index which is unique to other capture bead indices used in said assay and a multiplicity of the same capture element on said bead.

Embodiment 41: A capture bead for use in an assay conducted in an assay device said capture bead comprising a detectible label or a set of detectible labels attached to said bead which uniquely identifies the capture bead in said assay device wherein said detectible label is selected from visual, chemical, biological, audio or other means to evidence the presence of the information generated by the label.

Embodiment 42: A capture bead for use in an assay conducted in an assay device said capture bead comprising a detectible label or a set of detectible labels attached to said bead which uniquely identifies the capture bead in said assay device wherein said detectible label generates or can be stimulated to generate a color.

Embodiment 43: A capture bead for use in an assay conducted in an assay device said capture bead comprising a detectible label or a set of detectible labels attached to said bead which uniquely identifies the capture bead in said assay device wherein said detectible label generates or can be stimulated to emit light.

Embodiment 44: A capture bead for use in an assay conducted in an assay device said capture bead comprising a detectible label or a set of detectible labels attached to said bead which uniquely identifies the capture bead in said assay device wherein said detectible label comprises lettering.

Embodiment 45: A capture bead for use in an assay conducted in an assay device said capture bead comprising a detectible label or a set of detectible labels attached to said bead which uniquely identifies the capture bead in said assay device wherein said detectible label comprises numbers.

Embodiment 46: A capture bead for use in an assay conducted in an assay device said capture bead comprising a detectible label or a set of detectible labels attached to said bead which uniquely identifies the capture bead in said assay device wherein said detectible label comprises symbols.

Embodiment 47: A capture bead for use in an assay conducted in an assay device said capture bead comprising a detectible label or a set of detectible labels attached to said bead which uniquely identifies the capture bead in said assay device wherein said detectible label comprises one or more barcodes.

Embodiment 48: A capture bead for use in an assay conducted in an assay device said capture bead comprising a detectible label or a set of detectible labels attached to said bead which uniquely identifies the capture bead in said assay device wherein said detectible label comprises UPC codes.

Embodiment 49: A capture bead for use in an assay conducted in an assay device said capture bead comprising a detectible label or a set of detectible labels attached to said bead which uniquely identifies the capture bead in said assay device wherein said detectible label comprises one or more tags.

Embodiment 50: A capture bead for use in an assay conducted in an assay device said capture bead comprising a detectible label or a set of detectible labels attached to said bead which uniquely identifies the capture bead in said assay device wherein said detectible label comprises chemical signals.

Embodiment 51: A capture bead for use in an assay conducted in an assay device said capture bead comprising a detectible label or a set of detectible labels attached to said bead which uniquely identifies the capture bead in said assay device wherein said detectible label comprises quantum dots.

Embodiment 52: A capture bead for use in an assay conducted in an assay device said capture bead comprising a detectible label or a set of detectible labels attached to said bead which uniquely identifies the capture bead in said assay device wherein said detectible label comprises quantum dots and required instrumentation,

Embodiment 53: A capture bead for use in an assay conducted in an assay device said capture bead comprising a detectible label or a set of detectible labels attached to said bead which uniquely identifies the capture bead in said assay device wherein said detectible label comprises fluorescence.

Embodiment 54: A capture bead for use in an assay conducted in an assay device said capture bead comprising a detectible label or a set of detectible labels attached to said bead which uniquely identifies the capture bead in said assay device wherein said detectible label comprises mass spectroscopy.

Embodiment 55: A capture bead for use in an assay conducted in an assay device said capture bead comprising a detectible label or a set of detectible labels attached to said bead which uniquely identifies the capture bead in said assay device wherein said detectible label comprises nuclear magnetic spectra (e.g., a proton or C¹³ spectrum).

Embodiment 56: A capture bead for use in an assay conducted in an assay device said capture bead comprising a detectible label or a set of detectible labels attached to said bead which uniquely identifies the capture bead in said assay device wherein said detectible label comprises a biological signal or a biologically induced signal.

Embodiment 57: A capture bead for use in an assay conducted in an assay device said capture bead comprising a detectible label or a set of detectible labels attached to said bead which uniquely identifies the capture bead in said assay device wherein said detectible label comprises nucleic acids.

Embodiment 58: A capture bead for use in an assay conducted in an assay device said capture bead comprising a detectible label or a set of detectible labels attached to said bead which uniquely identifies the capture bead in said assay device wherein said detectible label comprises bioluminescence.

Embodiment 59: A capture bead for use in an assay conducted in an assay device said capture bead comprising a detectible label or a set of detectible labels attached to said bead which uniquely identifies the capture bead in said assay device wherein said detectible label comprises radio waves or electromagnetic signals.

Embodiment 60: A capture bead for use in an assay conducted in an assay device said capture bead comprising a detectible label or a set of detectible labels attached to said bead which uniquely identifies the capture bead in said assay device wherein said detectible label comprises Bluetooth.

Embodiment 61: A capture bead for use in an assay conducted in an assay device said capture bead comprising a detectible label or a set of detectible labels attached to said bead which uniquely identifies the capture bead in said assay device wherein said detectible label comprises RFID.

Embodiment 62: A capture bead for use in an assay conducted in an assay device said capture bead comprising a detectible label or a set of detectible labels attached to said bead which uniquely identifies the capture bead in said assay device wherein said detectible label comprises Bluetooth.

Embodiment 63: A capture bead for use in an assay conducted in an assay device said capture bead comprising a detectible label or a set of detectible labels attached to said bead which uniquely identifies the capture bead in said assay device wherein said detectible label comprises WiFi.

Embodiment 64: A capture bead for use in an assay conducted in an assay device said capture bead comprising a detectible label or a set of detectible labels attached to said bead which uniquely identifies the capture bead in said assay device wherein said detectible label comprises two or more labels selected from colors, codes, shapes, colors, inducible colors, lettering, numbering, symbols, bar codes, UPC codes, Bluetooth, WiFi, RFID, LEDs, bioluminescence, and nucleic acids.

Embodiment 65: A capture bead for use in an assay conducted in an assay device said capture bead comprising a detectible label or a set of detectible labels attached to said bead which uniquely identifies the capture bead in said assay device wherein said detectible label is selected from visual, chemical, biological, audio or other means to evidence the presence of the information generated by the label and a multiplicity of the same o capture bead index which is unique to other capture bead indices used in said assay.

Embodiment 66: A capture bead for use in an assay conducted in an assay device said capture bead comprising a detectible label or a set of detectible labels attached to said bead which uniquely identifies the capture bead in said assay device wherein said detectible label generates or can be stimulated to generate a color and a multiplicity of the same capture bead index which is unique to other capture bead indices used in said assay.

Embodiment 67: A capture bead for use in an assay conducted in an assay device said capture bead comprising a detectible label or a set of detectible labels attached to said bead which uniquely identifies the capture bead in said assay device wherein said detectible label generates or can be stimulated to emit light and a multiplicity of the same capture bead index which is unique to other capture bead indices used in said assay.

Embodiment 68: A capture bead for use in an assay conducted in an assay device said capture bead comprising a detectible label or a set of detectible labels attached to said bead which uniquely identifies the capture bead in said assay device wherein said detectible label comprises lettering and a multiplicity of the same capture bead index which is unique to other capture bead indices used in said assay.

Embodiment 69: A capture bead for use in an assay conducted in an assay device said capture bead comprising a detectible label or a set of detectible labels attached to said bead which uniquely identifies the capture bead in said assay device wherein said detectible label comprises numbers and a multiplicity of the same capture bead index which is unique to other capture bead indices used in said assay.

Embodiment 70: A capture bead for use in an assay conducted in an assay device said capture bead comprising a detectible label or a set of detectible labels attached to said bead which uniquely identifies the capture bead in said assay device wherein said detectible label comprises symbols and a multiplicity of the same capture bead index which is unique to other capture bead indices used in said assay.

Embodiment 71: A capture bead for use in an assay conducted in an assay device said capture bead comprising a detectible label or a set of detectible labels attached to said bead which uniquely identifies the capture bead in said assay device wherein said detectible label comprises one or more barcodes and a multiplicity of the same capture bead index which is unique to other capture bead indices used in said assay.

Embodiment 72: A capture bead for use in an assay conducted in an assay device said capture bead comprising a detectible label or a set of detectible labels attached to said bead which uniquely identifies the capture bead in said assay device wherein said detectible label comprises UPC codes and a multiplicity of the same capture bead index which is unique to other capture bead indices used in said assay.

Embodiment 73: A capture bead for use in an assay conducted in an assay device said capture bead comprising a detectible label or a set of detectible labels attached to said bead which uniquely identifies the capture bead in said assay device wherein said detectible label comprises one or more tags and a multiplicity of the same capture bead index which is unique to other capture bead indices used in said assay.

Embodiment 74: A capture bead for use in an assay conducted in an assay device said capture bead comprising a detectible label or a set of detectible labels attached to said bead which uniquely identifies the capture bead in said assay device wherein said detectible label comprises chemical signals and a multiplicity of the same capture bead index which is unique to other capture bead indices used in said assay.

Embodiment 75: A capture bead for use in an assay conducted in an assay device said capture bead comprising a detectible label or a set of detectible labels attached to said bead which uniquely identifies the capture bead in said assay device wherein said detectible label comprises optionally requiring suitable instrumentation quantum dots and a multiplicity of the same capture bead index which is unique to other capture bead indices used in said assay.

Embodiment 76: A capture bead for use in an assay conducted in an assay device said capture bead comprising a detectible label or a set of detectible labels attached to said bead which uniquely identifies the capture bead in said assay device wherein said detectible label comprises quantum dots and a multiplicity of the same capture bead index which is unique to other capture bead indices used in said assay.

Embodiment 77: A capture bead for use in an assay conducted in an assay device said capture bead comprising a detectible label or a set of detectible labels attached to said bead which uniquely identifies the capture bead in said assay device wherein said detectible label comprises fluorescence and a multiplicity of the same capture bead index which is unique to other oligonucleotide indices used in said assay.

Embodiment 78: A capture bead for use in an assay conducted in an assay device said capture bead comprising a detectible label or a set of detectible labels attached to said bead which uniquely identifies the capture bead in said assay device wherein said detectible label comprises mass spectroscopy and a multiplicity of the same oligonucleotide index which is unique to other capture bead indices used in said assay.

Embodiment 79: A capture bead for use in an assay conducted in an assay device said capture bead comprising a detectible label or a set of detectible labels attached to said bead which uniquely identifies the capture bead in said assay device wherein said detectible label comprises nuclear magnetic spectra (e.g., a proton or C¹³ spectrum) and a multiplicity of the same capture bead index which is unique to other capture bead indices used in said assay.

Embodiment 80: A capture bead for use in an assay conducted in an assay device said capture bead comprising a detectible label or a set of detectible labels attached to said bead which uniquely identifies the capture bead in said assay device wherein said detectible label comprises a biological signal or a biologically induced signal and a multiplicity of the same capture bead index which is unique to other capture bead indices used in said assay.

Embodiment 81: A capture bead for use in an assay conducted in an assay device said capture bead comprising a detectible label or a set of detectible labels attached to said bead which uniquely identifies the capture bead in said assay device wherein said detectible label comprises nucleic acids and a multiplicity of the same capture bead index which is unique to other capture bead indices used in said assay.

Embodiment 82: A capture bead for use in an assay conducted in an assay device said capture bead comprising a detectible label or a set of detectible labels attached to said bead which uniquely identifies the capture bead in said assay device wherein said detectible label comprises bioluminescence and a multiplicity of the same capture bead index which is unique to other capture bead indices used in said assay.

Embodiment 83: A capture bead for use in an assay conducted in an assay device said capture bead comprising a detectible label or a set of detectible labels attached to said bead which uniquely identifies the capture bead in said assay device wherein said detectible label comprises radio waves or electromagnetic signals and a multiplicity of the same capture bead index which is unique to other capture bead indices used in said assay.

Embodiment 84: A capture bead for use in an assay conducted in an assay device said capture bead comprising a detectible label or a set of detectible labels attached to said bead which uniquely identifies the capture bead in said assay device wherein said detectible label comprises Bluetooth and a multiplicity of the same capture bead index which is unique to other capture bead indices used in said assay.

Embodiment 85: A capture bead for use in an assay conducted in an assay device said capture bead comprising a detectible label or a set of detectible labels attached to said bead which uniquely identifies the capture bead in said assay device wherein said detectible label comprises RFID and a multiplicity of the same capture bead index which is unique to other capture bead indices used in said assay.

Embodiment 86: A capture bead for use in an assay conducted in an assay device said capture bead comprising a detectible label or a set of detectible labels attached to said bead which uniquely identifies the capture bead in said assay device wherein said detectible label comprises Bluetooth and a multiplicity of the same capture bead index which is unique to other capture bead indices used in said assay.

Embodiment 87: A capture bead for use in an assay conducted in an assay device said capture bead comprising a detectible label or a set of detectible labels attached to said bead which uniquely identifies the capture bead in said assay device wherein said detectible label comprises WiFi and a multiplicity of the same capture bead index which is unique to other capture bead indices used in said assay.

Embodiment 88: A capture bead for use in an assay conducted in an assay device said capture bead comprising a detectible label or a set of detectible labels attached to said bead which uniquely identifies the capture bead in said assay device wherein said detectible label comprises two or more labels selected from colors, codes, shapes, colors, inducible colors, lettering, numbering, symbols, bar codes, UPC codes, Bluetooth, WiFi, RFID, bioluminescence, and nucleic acids. and a multiplicity of the same capture bead index which is unique to other capture bead indices used in said assay.

Embodiment 89: A capture bead for use in an assay conducted in an assay device said capture bead comprising a detectible label or a set of detectible labels attached to said bead which uniquely identifies the capture bead in said assay device wherein said detectible label is selected from visual, chemical, biological, audio or other means to evidence the presence of the information generated by the label, a multiplicity of the same capture bead index which is unique to other capture bead indices used in said assay and a multiplicity of the same or different capture elements attached to at least a portion of said indices optionally through a cleavable or non-cleavable linker.

Embodiment 90: A capture bead for use in an assay conducted in an assay device said capture bead comprising a detectible label or a set of detectible labels attached to said bead which uniquely identifies the capture bead in said assay device wherein said detectible label generates or can be stimulated to generate a color, a multiplicity of the same capture bead index which is unique to other capture bead indices used in said assay, and a multiplicity of the same or different capture elements attached to at least a portion of said indices optionally through a cleavable or non-cleavable linker.

Embodiment 91: A capture bead for use in an assay conducted in an assay device said capture bead comprising a detectible label or a set of detectible labels attached to said bead which uniquely identifies the capture bead in said assay device wherein said detectible label generates or can be stimulated to emit light, a multiplicity of the same capture bead index which is unique to other capture bead indices used in said assay, and a multiplicity of the same or different capture elements attached to at least a portion of said indices optionally through a cleavable or non-cleavable linker.

Embodiment 92: A capture bead for use in an assay conducted in an assay device said capture bead comprising a detectible label or a set of detectible labels attached to said bead which uniquely identifies the capture bead in said assay device wherein said detectible label comprises lettering and a multiplicity of the same capture bead index which is unique to other capture bead indices used in said assay, and a multiplicity of the same or different capture elements attached to at least a portion of said indices optionally through a cleavable or non-cleavable linker.

Embodiment 93: A capture bead for use in an assay conducted in an assay device said capture bead comprising a detectible label or a set of detectible labels attached to said bead which uniquely identifies the capture bead in said assay device wherein said detectible label comprises numbers, a multiplicity of the same capture bead index which is unique to other capture bead indices used in said assay, and a multiplicity of the same or different capture elements.

Embodiment 94: A capture bead for use in an assay conducted in an assay device said capture bead comprising a detectible label or a set of detectible labels attached to said bead which uniquely identifies the capture bead in said assay device wherein said detectible label comprises symbols, a multiplicity of the same capture bead index which is unique to other capture bead indices used in said assay, and a multiplicity of the same or different capture elements attached to at least a portion of said indices, optionally via a linker that may be a same or different cleavable or non-cleavable linkers.

Embodiment 95: A capture bead for use in an assay conducted in an assay device said capture bead comprising a detectible label or a set of detectible labels attached to said bead which uniquely identifies the capture bead in said assay device wherein said detectible label comprises one or more barcodes, a multiplicity of the same capture bead index which is unique to other capture bead indices used in said assay, and a multiplicity of the same or different capture element attached to at least a portion of said indices optionally through a cleavable or non-cleavable linker.

Embodiment 96: A capture bead for use in an assay conducted in an assay device said capture bead comprising a detectible label or a set of detectible labels attached to said bead which uniquely identifies the capture bead in said assay device wherein said detectible label comprises UPC codes, a multiplicity of the same capture bead index which is unique to other capture bead indices used in said assay, and a multiplicity of the same or different capture element attached to at least a portion of said indices optionally through a cleavable or non-cleavable linker.

Embodiment 97: A capture bead for use in an assay conducted in an assay device said capture bead comprising a detectible label or a set of detectible labels attached to said bead which uniquely identifies the capture bead in said assay device wherein said detectible label comprises one or more tags, a multiplicity of the same capture bead index which is unique to other capture bead indices used in said assay, and a multiplicity of the same or different capture elements attached to at least a portion of said indices optionally through a cleavable or non-cleavable linker.

Embodiment 98: A capture bead for use in an assay conducted in an assay device said capture bead comprising a detectible label or a set of detectible labels attached to said bead which uniquely identifies the capture bead in said assay device wherein said detectible label comprises chemical signals, a multiplicity of the same capture bead index which is unique to other capture bead indices used in said assay, and a multiplicity of the same or different capture elements attached to at least a portion of said indices optionally through a cleavable or non-cleavable linker.

Embodiment 99: A capture bead for use in an assay conducted in an assay device said capture bead comprising a detectible label or a set of detectible labels attached to said bead which uniquely identifies the capture bead in said assay device wherein said detectible label comprises optionally requiring suitable instrumentation quantum dots, a multiplicity of the same capture bead indices which is unique to other capture bead indices used in said assay, and a multiplicity of the same or different capture elements.

Embodiment 100: A capture bead for use in an assay conducted in an assay device said capture bead comprising a detectible label or a set of detectible labels attached to said bead which uniquely identifies the capture bead in said assay device wherein said detectible label comprises quantum dots, a multiplicity of the same capture bead index which is unique to other capture bead indices used in said assay, and a multiplicity of the same or different capture elements attached to at least a portion of said indices optionally through a cleavable or non-cleavable linker.

Embodiment 101: A capture bead for use in an assay conducted in an assay device said capture bead comprising a detectible label or a set of detectible labels attached to said bead which uniquely identifies the capture bead in said assay device wherein said detectible label comprises fluorescence, a multiplicity of the same capture bead index which is unique to other capture bead indices used in said assay, and a multiplicity of the same or different capture elements attached to at least a portion of said indices optionally through a cleavable or non-cleavable linker.

Embodiment 102: A capture bead for use in an assay conducted in an assay device said capture bead comprising a detectible label or a set of detectible labels attached to said bead which uniquely identifies the capture bead in said assay device wherein said detectible label comprises mass spectroscopy, a multiplicity of the same capture bead index which is unique to other capture bead indices used in said assay, and a multiplicity of the same or different capture elements attached to at least a portion of said indices optionally through a cleavable or non-cleavable linker.

Embodiment 103: A capture bead for use in an assay conducted in an assay device said capture bead comprising a detectible label or a set of detectible labels attached to said bead which uniquely identifies the capture bead in said assay device wherein said detectible label comprises nuclear magnetic spectra (e.g., a proton or C¹³ spectrum), a multiplicity of the same capture bead index which is unique to other capture bead indices used in said assay, and a multiplicity of the same or different capture elements attached to at least a portion of said indices optionally through a cleavable or non-cleavable linker.

Embodiment 104: A capture bead for use in an assay conducted in an assay device said capture bead comprising a detectible label or a set of detectible labels attached to said bead which uniquely identifies the capture bead in said assay device wherein said detectible label comprises a biological signal or a biologically induced signal, a multiplicity of the same capture bead index which is unique to other capture bead indices used in said assay, and a multiplicity of the same or different capture elements attached to at least a portion of said indices optionally through a cleavable or non-cleavable linker.

Embodiment 105: A capture bead for use in an assay conducted in an assay device said capture bead comprising a detectible label or a set of detectible labels attached to said bead which uniquely identifies the capture bead in said assay device wherein said detectible label comprises nucleic acids, a multiplicity of the same capture bead index which is unique to other capture bead indices used in said assay, and a multiplicity of the same or different capture elements attached to at least a portion of said indices optionally through a cleavable or non-cleavable linker.

Embodiment 106: A capture bead for use in an assay conducted in an assay device said capture bead comprising a detectible label or a set of detectible labels attached to said bead which uniquely identifies the capture bead in said assay device wherein said detectible label comprises bioluminescence, a multiplicity of the same capture bead index which is unique to other capture bead indices used in said assay, and a multiplicity of the same or different capture elements attached to at least a portion of said indices optionally through a cleavable or non-cleavable linker.

Embodiment 107: A capture bead for use in an assay conducted in an assay device said capture bead comprising a detectible label or a set of detectible labels attached to said bead which uniquely identifies the capture bead in said assay device wherein said detectible label comprises radio waves or electromagnetic signals, a multiplicity of the same capture bead index which is unique to other capture bead indices used in said assay, and a multiplicity of the same or different capture elements attached to at least a portion of said indices optionally through a cleavable or non-cleavable linker.

Embodiment 108: A capture bead for use in an assay conducted in an assay device said capture bead comprising a detectible label or a set of detectible labels attached to said bead which uniquely identifies the capture bead in said assay device wherein said detectible label comprises Bluetooth, a multiplicity of the same capture bead index which is unique to other capture bead indices used in said assay, and a multiplicity of the same or different capture elements attached to at least a portion of said indices optionally through a cleavable or non-cleavable linker.

Embodiment 109: A capture bead for use in an assay conducted in an assay device said capture bead comprising a detectible label or a set of detectible labels attached to said bead which uniquely identifies the capture bead in said assay device wherein said detectible label comprises RFID, a multiplicity of the same capture bead index which is unique to other capture bead indices used in said assay, and a multiplicity of the same or different capture elements attached to at least a portion of said indices optionally through a cleavable or non-cleavable linker.

Embodiment 110: A capture bead for use in an assay conducted in an assay device said capture bead comprising a detectible label or a set of detectible labels attached to said bead which uniquely identifies the capture bead in said assay device wherein said detectible label comprises Bluetooth, a multiplicity of the same capture bead index which is unique to other capture bead indices used in said assay, and a multiplicity of the same or different capture elements attached to at least a portion of said indices optionally through a cleavable or non-cleavable linker.

Embodiment 111: A capture bead for use in an assay conducted in an assay device said capture bead comprising a detectible label or a set of detectible labels attached to said bead which uniquely identifies the capture bead in said assay device wherein said detectible label comprises WiFi, a multiplicity of the same capture bead index which is unique to other capture bead indices used in said assay, and a multiplicity of the same or different capture elements attached to at least a portion of said indices optionally through a cleavable or non-cleavable linker.

Embodiment 112: A capture bead for use in an assay conducted in an assay device said capture bead comprising a detectible label or a set of detectible labels attached to said bead which uniquely identifies the capture bead in said assay device wherein said detectible label comprises two or more labels selected from colors, codes, shapes, colors, inducible colors, lettering, numbering, symbols, bar codes, UPC codes, Bluetooth, WiFi, RFID, bioluminescence, and nucleic acids, a multiplicity of the same capture bead index which is unique to other capture bead indices used in said assay, and a multiplicity of the same or different capture elements attached to at least a portion of said indices optionally through a cleavable or non-cleavable linker.

Embodiment 113: A method for associating a capture bead to a specific examination area in a mapped assay device which method comprises coding each of said capture beads with an optically detectible label or a set of optically detectible labels unique to that bead and further coding the capture bead with an oligonucleotide that uniquely codes the label or set of labels on said bead; adding a capture bead to at least a portion of the examination areas in said device; and associating each unique labeled bead to each examination area in said device.

Embodiment 114: A method for associating a capture bead to a specific examination area in a mapped assay device which method comprises coding each of said capture beads with an optically detectible label or a set of optically detectible labels unique to that bead; adding a capture bead to at least a portion of the examination areas in said device; associating each unique labeled capture bead to each examination area in said device; and memorializing each position of each capture bead to the specific examination area on the mapped assay device.

Embodiment 115: The method of Embodiments 113 and 114 wherein the label is as defined in any one of Embodiments 41 to 66.

Embodiment 116: The method of Embodiment 115 wherein the capture bead further comprises a capture bead index and a capture element as defined in any one of embodiments 89 through 113 attached thereto.

Embodiment 117: A method for conducting an assay in at least a portion of a multiplicity of examination areas in a mapped assay device wherein each of said examination areas comprises a cell, a perturbation bead comprising a perturbation element, and a perturbation oligonucleotide which is unique to said compound, and an assay solution, said method comprises:
a) conducting an assay in at least a portion of a multiplicity of examination areas in a mapped assay device wherein each of said examination areas comprises a cell, a perturbation bead comprising a perturbation element, and a perturbation oligonucleotide that is unique to said compound, and an assay solution;
b) including in each of said examination areas a capture bead which comprises:
   an optically detectible label or a set of optically detectible labels attached to said bead which uniquely identifies the capture bead;
   a multiplicity of the same oligonucleotide index which is unique to said capture bead and is attached to thereto optionally through a linker wherein said index codes for the unique label or set of labels; and
   a multiplicity of the same capture element attached to the oligonucleotide index;
c) correlating and memorializing the optically detectible label or set of labels in each examination area by associating the unique optically detectible labels on said capture bead to said mapped assay device thereby providing for a 1:1 relationship between each capture bead in each of the examination areas;
d) inducing a perturbation by releasing at least a portion of the perturbation elements from said perturbation bead thereby allowing said elements to contact said cell and induce functional changes in said cell which comprises at least a change evidenced by one or more perturbation components expressed by said cell;
e) releasing the unique perturbation oligonucleotide from said bead and capturing said oligonucleotide onto the capture bead;
f) lysing said cell to release said perturbation components into said assay solution and capturing at least one of said components onto the capture bead index or the perturbation oligonucleotide;
g) sequencing the oligonucleotide comprising the oligonucleotide index, the perturbation oligonucleotide, and the perturbation component; and
h) correlating the oligonucleotide index to a unique label or set of labels to a specific examination area by reference to the memorialized correlation in c) above thereby identifying the particular examination area from which the perturbation component was retrieved.

Embodiment 118: The method of Embodiment 117 which further comprises:
i) obtaining images of each of the examination areas during the assay;
j) correlating the images to the specific examination area defined in H) above; and
k) evaluating changes in cellular morphology during the assay as a result of the perturbation of the cell.

Embodiment 119: The multiplicity of the same capture element on the capture bead is a receptor or ligand for the perturbation component to be captured.

Embodiment 120: The multiplicity of the same capture element on the capture bead is an antibody or an antibody binding fragment for the perturbation compound.

Embodiment 121: The multiplicity of the same capture element on the capture bead is an enzyme or enzyme receptor for the perturbation component.

Embodiment 122: The multiplicity of the same capture element on the capture bead is a receptor for a given cytokine or chemokine.

Embodiment 123: The multiplicity of the same capture element on the capture bead comprises avidin/streptavidin that captures perturbation components that have been modified to contain a biotin group.

Embodiment 124: The multiplicity of the same capture element on the capture bead comprises biotin that captures perturbation components that have been modified to contain an avidin/streptavidin group.

Embodiment 125: The multiplicity of the same oligonucleotide index and the multiplicity of the same oligonucleotide capture element are attached to each other either directly or through a releasable linker to the capture bead.

Embodiment 126: The multiplicity of the same oligonucleotide capture element on said capture bead is poly-T.

Embodiment 127: A library or registry comprising a recitation of each of the labels or set of detectible labels that code for a unique capture bead and the specific examination area in the assay device where each of said capture beads are located.

Embodiment 128: A capture bead comprising a detectible label or a set of detectible labels that uniquely associates the capture bead to a specific examination area wherein said capture bead comprises additional information that is related to the assay to be conducted in that examination area including the conditions of the assay, the change in functionality of the cell during the assay, the synthetic reactions used in at least one step of the compound synthesis, the identity of one or more reaction conditions used during synthesis, the technician conducting the assay, the date of the assay, the pH of the assay solution, or combinations thereof.

Embodiment 129: The unique optically detectible label on each of said capture beads in each examination area is recorded for example by photography, videography, bar codes, or QR codes.

Embodiment 130: A capture bead presented by Formula I below:

(W)ᵣ—CB-(L-X-Q-Q¹)ₙ. (I)

where:
CB is a capture bead;
L is a linker;
W is an optically detectible label or a set of optically detectible labels that uniquely identify the capture bead;
X is a bond or a capture element;
Q is a capture bead index that codes the optically detectible label or set of optically detectible labels on the capture bead;
Q¹ is a capture element which is independently selected for each of the n L-X-Q-Q¹ groups;
n represents the multiplicity of such (linker-Q-Q¹) groups bound to the bead, and
r is an integer from 1 to 10.

Embodiment 131: The capture bead of Embodiment 130 where W is a label as defined in any one of Embodiments 41 to 66.

Embodiment 132: The capture bead of Embodiment 130 where the capture bead further comprises a capture bead index and a capture element as defined in any one of embodiments 89 through 113.

Embodiment 133: The capture bead of Embodiment 130 where the attachment of Q to Q¹ is either a direct linkage or through a linker.

Embodiment 134: The capture bead of Formula I can include Formula I-A, which is described as in Formula I-A (below):

(W)ᵣ—CB-[L′-X-Q-Q¹-PC]ₙ (I-A)

where:
Q is a capture bead oligonucleotide;
X is a bond or a capture element;
Q¹ is a capture element wherein when X is not a bond, both X and Q¹ are independently capture elements wherein the capture element on each of Q¹ and X are complementary to a corresponding functional group on a target;
PC is a perturbation component;
L′ is a releasable linker;
and n, r, and W are as defined above.

Embodiment 134: An oligonucleotide represented by the Formula I-B:

A-X¹-Q-Q¹-PC I-B

where:
PC is a perturbation component;
Q is the capture index;
Q¹ is a capture element complementary to a corresponding functional group on PC;
X¹ is a capture element complementary to a corresponding functional group on A; and
A is a perturbation component or the perturbation oligonucleotide.

Embodiment 135: The oligonucleotide of Embodiment 134, where PC is mRNA and Q¹ is poly-T.

Embodiment 136: The oligonucleotide of Embodiment 134, where A is a perturbation oligonucleotide and X¹ is a complementary group to a functional group on said perturbation oligonucleotide.

Embodiment 137: The oligonucleotide of Embodiment 136, where A is a nucleic acid other than mRNA

Embodiment 138: A method for preparing a population of beads wherein each bead in said population comprises a unique label or a set of labels and an oligonucleotide specific for that label or set of labels which method comprises:
a) selecting a population of beads which are functionalized with an oligonucleotide binding site;
b) splitting said beads in approximately equal numbers into a multiplicity of reaction vessels wherein the number of reaction vessels ranges from 3 to 12;
c) into each reaction vessel, apply a first label wherein the label used in each vessel is different from the other vessels;
d) attach an oligonucleotide strand to said oligonucleotide binding site;
e) pooling the beads and mixing until homogeneous;
f) repeat the process of b) and e) a sufficient number of times such that each of the beads in said population is uniquely labeled and each bead comprises a unique oligonucleotide which comprises oligonucleotide strands attached to the prior oligonucleotide strands for each of the separate reaction steps.

What is claimed is:

1. A capture bead for use in an assay, said capture bead comprising:
a) an optically detectible label or a set of optically detectible labels, attached to said capture bead, which uniquely identifies the capture bead;
b) a multiplicity of a capture bead index that codes for the detectible label or set of detectible labels and that is attached to the capture bead by a linker; and
c) a multiplicity of one or more capture elements, wherein each of the one or more capture elements is attached to a capture bead index of the multiplicity of the capture bead index.

2. The capture bead of claim 1, wherein said detectible label or said set of detectible labels is optically detectible.

3. The capture bead of claim 2, wherein said optically detectible label comprises one or more of an image, a code, a shape, a color, an inducible color, lettering, numbering, a symbol, a bar code, a UPC code, or optically detectible materials.

4. The capture bead of claim 1, wherein the linker is a releasable linker.

5. The capture bead of claim 1, wherein the capture bead index comprises from about 6 to about 2,000 nucleotides.

6. The capture bead of claim 1, wherein each capture element, of the one or more capture elements, is selected from an oligonucleotide, receptor, or ligand, wherein the capture element corresponds to a target perturbation component or to a perturbation oligonucleotide that codes for a perturbation element.

7. A mapped assay device comprising a multiplicity of examination areas, wherein each examination area, of at least a portion of the multiplicity of examination areas, comprises at least one capture bead as defined in claim 1, and wherein said mapped assay device is configured to correlate a location of the examination area to the at least one capture bead in the examination area.

8. A register, for the assay device of claim 7, indicating, for each optically detectable label or set of optically detectible labels on each of the at least one capture beads:
the optically detectable label or set of optically detectible labels; and
a corresponding examination area, of the multiplicity of examination areas, from which the optically detectible label or set of optically detectible labels was detected.

9. The register of claim 8 comprising one or more images that record each optically detectible label or set of optically detectible labels in the corresponding examination area.

10. The capture bead of claim 1, wherein the capture bead is represented by Formula I:
(W)ᵣ—CB-(L-X-Q-Q¹)ₙ (I)
where:
CB is a bead;
L is a linker;
W is an optically detectible label or a set of optically detectible labels that identify the capture bead;
X is a bond or a capture element;
Q is a capture bead index that codes for the optically detectible label or set of optically detectible labels;
Q¹ is a capture element that is independently selected for each of the n L-X-Q-Q¹ groups;
n is a number of such (L-X-Q-Q¹) groups bound to the bead, and
r is an integer from 1 to 100.

11. The capture bead of claim 10, wherein n ranges from 10 to 6.02×10¹⁷.

12. The capture bead of claim 10, wherein L-X-Q-Q¹ is the same for each of the n L-X-Q-Q¹ groups.

13. The capture bead of claim 12, wherein X and Q¹ are binding elements configured to capture different targets.

14. The capture bead of claim 10, where each X and Q¹ are independently selected, for each of the n L-X-Q-Q¹ groups, from up to 15 capture elements.

15. The capture bead of claim 10, wherein L is a releasable linker.

16. The capture bead of claim 10, wherein one of X or Q¹ is configured to capture a perturbation oligonucleotide that codes for a perturbation element, and the other is configured to capture one or more perturbation components released from a cell.

17. A capture bead represented by Formula I-A:
(W)ᵣ—CB-[L′-X-Q-Q¹-Y]ₙ (I-A)
where:
W is an optically detectible label or a set of optically detectible labels that identify the capture bead;
Q is a capture bead index oligonucleotide;
X is a bond or a capture element;
Q¹ is a capture element wherein both X and Q¹ are independently capture elements when X is a capture element wherein the capture element on both Q¹ and X are complementary to a corresponding functional group on a target;
Y is one or more of a perturbation oligonucleotide PO that codes for a perturbation element PE or a perturbation component PC released from a cell;
L′ is a linker; and
n is a quantity of the (L-X-Q-Q¹-PC) groups bound to the bead, and
r is an integer from 1 to 100.

18. An oligonucleotide represented by the Formula I-B:
A-X¹-Q-Q¹-PC (I-B)
where:
PC is a perturbation component released from a cell following exposure of the cell to a perturbation element;
Q is a capture bead index;
Q¹ is a capture element complementary to a corresponding functional group on PC;
X¹ is a capture element complementary to a corresponding functional group on A; and
A is a PC or a perturbation oligonucleotide that codes for a perturbation element.

19. The oligonucleotide of claim 18, wherein PC is mRNA and Q¹ comprises a poly-T.

20. The oligonucleotide of claim 19, wherein A is a perturbation oligonucleotide and X¹ is a complementary group to a functional group on said perturbation oligonucleotide.

21. The oligonucleotide of claim 19, wherein A is a nucleic acid other than mRNA.

22. A method for identifying a specific examination area, or a multiplicity of examination areas in a mapped assay device, in which a perturbation element was released, which method comprises:
a) providing, in each examination area of the multiplicity of examination areas in the mapped assay device:
a cell;
a corresponding perturbation bead, of a multiplicity of perturbation beads each comprising a perturbation element, of one or more perturbation elements, and a perturbation oligonucleotide, of one or more perturbation oligonucleotides, that codes for the perturbation element; and
a corresponding capture bead, of a multiplicity of capture beads, comprising:
an optically detectible label or a set of optically detectible labels, attached to said capture bead, which uniquely identifies the capture bead;
a multiplicity of a capture bead index that codes for the detectible label or set of detectible labels and that is attached to the capture bead by a linker; and
a multiplicity of one or more capture elements, wherein each of the one or more capture elements is attached to a capture bead index of the multiplicity of the capture bead index;
c) memorializing information correlating, for each corresponding capture bead, of the multiplicity of capture beads, the optically detectible label or set of optically detectible labels thereon to a location of the corresponding examination area, thereby providing for a 1:1 relationship between each capture bead and each examination area of the multiplicity of examination areas;
d) in each examination area of the multiplicity of examination areas, causing release of at least a portion of the perturbation elements from said perturbation bead, thereby allowing said perturbation elements to contact said cell;
e) in each examination area of the portion, causing release of the perturbation oligonucleotide from said perturbation bead to allow for capturing said perturbation oligonucleotide by the capture bead;
f) in each examination area of the portion, lysing said cell to allow for release of perturbation components, wherein one or more of the capture element or the perturbation oligonucleotide are configured to capture a perturbation component of said perturbation components;
g) sequencing oligonucleotides, resulting from said capture in f), comprising the capture bead index, the perturbation oligonucleotide, and the perturbation component; and
h) identifying the specific examination area by correlating a sequenced capture bead index, from a sequenced oligonucleotide, to the optically detectible label or set of optically detectable labels correlated to the specific examination area by reference to the memorialized information.

23. The method of claim 22, further comprising:
i) obtaining images of each of the examination areas during the assay;
j) correlating an image, of the images, to the specific examination area; and
k) evaluating the image for changes in cellular morphology during the assay after release of the perturbation element.

24. The method of claim 22, wherein the perturbation element is a compound.

25. The method of claim 22, wherein the perturbation element is selected from one or more of:
antibodies, immune cells, siRNA, viruses, bacteria, fungi, or
a change in one or more assay conditions such as buffers, salts, pH, temperature, nutrients, oxygen levels, oxidizing agents, or physical stress.

26. The method of claim 22, wherein the corresponding capture bead is provided in the examination area before or during the assay.

27. The method of claim 22, wherein each corresponding capture bead, of the multiplicity of capture beads, is represented by Formula I-A:
(W)ᵣ—CB-[L′-X-Q-Q-PC]ₙ (I-A)
where:
Q is a capture bead index;
X is a bond or a second capture element on Q;
Q¹ is a capture element wherein both X and Q¹ are independently capture elements when X is a capture element wherein the capture element on both Q¹ and X are complementary to a corresponding functional group on a target;
PC is a perturbation element of the perturbation components;
L′ is a linker;
W is an optically detectible label or a set of optically detectible labels that uniquely identify the capture bead;
n represents the multiplicity of such (L′-X-Q-Q¹-PC) groups bound to the bead, and
r is an integer from 1 to 100.

28. The method of claim 27, wherein cleaving the linker on said corresponding capture bead is conducted after causing release of the perturbation elements but prior to lysing the cell, such that the capturing of the perturbation oligonucleotide occurs on the capture bead, but the capture of the perturbation component released from the lysed cell occurs in solution.

29. The method of claim 27, wherein cleaving the linker is conducted prior to or concurrent with causing release of the perturbation oligonucleotide such that the capture of perturbation components and/or the perturbation oligonucleotide occurs in solution.
